(19) United States
(12) Patent Application Publication
(10) Pub. No.: US 2003/0170656 A1
(43) Pub. Date: Sep. 11, 2003

(54) Method of screening for factors that modulate gene expression

(75) Inventors: Hui Cen, Oakland, CA (US); Jaime Escobedo, Alamo, CA (US); Lewis Williams, Tiburon, CA (US)

Correspondence Address: KIMBERLIN MORLEY, ESQ., CHIRON CORPORATION, INTELLECTUAL PROPERTY - R440, P.O. BOX 8097, EMERYVILLE, CA, 94662-8097, US

(73) Assignee: Chiron Corporation, Emeryville, CA

(21) Appl. No.: 10/174,152

(22) Filed: Jun. 18, 2002

Related U.S. Application Data
(63) Continuation of application No. 08/729,143, filed on Oct. 11, 1996.
(60) Provisional application No. 60/005,499, filed on Oct. 16, 1995.

(51) Int. Cl. C12Q 1/68; G01N 33/53; G01N 33/567

(52) U.S. Cl. 435/6; 435/7.2

(57) ABSTRACT

The invention describes a method of screening for factors that modulate gene expression, by detection of factors including factors that stimulate or inhibit cell growth, differentiation or proliferation, factors involved in one or more signal transduction pathways, or factors involved in protein-protein interactions. Identification of such factors that modulate gene expression is accomplished by detection of an intracellular event mediated by the factor. The intracellular event can be an increase or decrease in transcription or translational activities. Detection of the intracellular event is accomplished by any highly sensitive assay targeting a transcription or translation product, including, for example, a bDNA assay to the nascent transcript induced by the factor.

### CROSS-REFERENCE TO RELATED APPLICATION

This application is related to provisional patent application serial No. 60/005,499, filed Oct. 16, 1995, from which priority is claimed under 35 U.S.C. §119(e)(1) and which is incorporated herein by reference in its entirety.

### FIELD OF THE INVENTION

The present invention relates to a method of screening for factors that modulate gene expression. The factors identifiable by the invention include those that stimulate or inhibit growth, differentiation, or proliferation of cells, and thus include factors that are involved in signal transduction pathways, other intracellular signaling, and can exert transcriptional and translational effects. Essentially, the effect of these factors on a responding cell is detected by detecting an intracellular event in the responding cell. The intracellular event may be an increase or decrease in transcriptional or translational activities, or an increase or decrease in signal transduction activities or other signaling activities. Detection of the intracellular event is accomplished by any appropriate means sufficiently sensitive to detect such intracellular response including, for example, bDNA assay, RNase protection assay, or RT-PCR detection means.

### BACKGROUND OF THE INVENTION

Conventional methods of identification of factors that modulate gene expression typically require relatively large amounts of the factor to be tested and a large number of responding cells in order to generate a detectable signal. These methods are generally not applicable to screening of factors that are naturally produced in cells in small or minute quantities. Since most biological factors are generated by cells in small quantities, it would be advantageous to design a sensitive assay to allow the detection or identification of factors that are produced in small amounts and to study the direct or indirect effect of such factors on a small number of responding cells. Additionally, it would be advantageous to design an assay for screening factors that allows screening of many factors quickly and accurately, and also which provides the opportunity to screen primary cell culture, as opposed to requiring cells transformed with reporter constructs for detection of modulating factors.

### SUMMARY OF THE INVENTION

The invention is a method of high throughput screening for a factor that modulates gene expression comprising providing a small amount of a candidate factor, providing a small amount of responding cells, contacting a responding cell with a candidate factor, wherein the responding cell is capable of responding to a factor that modulates gene expression by exhibiting an early intracellular event; and detecting directly the early intracellular event. The invention also comprises a detection facilitated by hybridization of a polynucleotide sequence to a target polynucleotide, provides that the target polynucleotide can be an mRNA transcript, that the polynucleotide sequence that hybridizes to a target polynucleotide can be selected from the group consisting of synthetic DNA, synthetic RNA, reverse transcription primers, polymerase chain reaction primers, and an RNase protection assay probe. Also the polynucleotide sequence that hybridizes to the target polypeptide can be the synthetic DNA, branched DNA. The candidate factor can be a polypeptide factor, small molecule factor, and a polynucleotide factor. The method can also comprise co-culturing a producing cell and a responding cell.

In accordance to a further object of the present invention, there is provided a growth factor, a differentiation factor, a hormone, a cytokine, a transcription factor, an inhibitory factor, a ligand and/or a receptor, or an antagonist to a receptor, produced by the methods described above.

In accordance to a further object of the present invention, there is provided a polypeptide of the sequence of SEQ ID NO. 2, that exhibits growth factor activity as demonstrated by induction of c-fos transcription.

In accordance to a further object of the present invention, there is provided a polypeptide having the sequence of SEQ ID No. 2.

In accordance to a further object of the present invention, there is provided a polynucleotide sequence of SEQ ID No. 1 connected to a heterologous polynucleotide sequence.

Among other factors, the invention is designed to screen for factors that modulate gene expression and is a fast and efficient way to screen a large amount of candidate factors in an assay in which only a small amount of any one candidate factor is required for a detectable effect to occur in a small number of cells.

### DETAILED DESCRIPTION OF THE PREFERRED EMBODIMENTS

The invention described herein draws on previously published work and pending patent applications. By way of example, such work consists of scientific papers, patents or pending patent applications. All such published work cited herein are hereby incorporated by reference.

The inventors have established an in vivo method of screening for factors that modulate gene expression. Such factors can be small molecules or polypeptide factors, and the factors can be stimulatory or inhibitory, for example. The genes that are modulated can be any gene, the expression of which can be modulated, and the modulation of the expression of which is an indication of a desirable change in the cell. For example, where a growth factor is sought, the gene the can be modulated can be a gene associated with cell growth, for example the c-fos gene, and the modulation of the gene can be an increase in the gene transcript. The desirable change in the cell can be cell growth that occurs in response to administration of the modulatory factor.

Detection of the gene expression modulatory effects of a factor can be made by any sufficiently sensitive means, and may be targeted to transcriptional modulation, or translational modulation. Transcriptional modulation can be detected by detecting changes in levels of transcripts of target genes or genes designated as indicators of larger effects, such as, for example, cell growth, cell differentiation, growth arrest, inflammation response, a signalling pathway, and expression of other genes.

Definitions

“A factor that modulates gene expression” as used herein refers to a compound, presently known or unknown, that is capable of causing the manifestation of an intracellular event in a responding cell. Such intracellular event may be an increase or decrease in transcriptional or translational activity as well as signal transduction activity. The compound can be a protein, a polypeptide, a peptide, a peptoid or other small molecule, and can be naturally occurring or synthetically made. Such a factor can be a stimulatory factor or an inhibitory factor. The factor can also be a ligand or a receptor which, upon contact of one to the other, is capable of signal transduction in a cell. The factor can be a natural product of a producing cell or can be the product of expression of a producing cell that is transformed by a polynucleotide encoding the factor. In each instance, the factor can be a secreted factor, or a factor expressed on the surface of the cell, or a nuclear-acting factor such as a transcription factor, or a factor that is released upon lysis of the cell. The factor herein may act directly or indirectly, and may modulate the gene of interest, a regulatory sequence controlling the expression of the gene of interest, or may modulate the expression of another gene that then modulates the gene of interest.

The term “directly” as used herein with regard to detection of an early intracellular event, means that the detection means directly binds to a transcript, or other target, which binding may be amplified for detection purposes, but which amplification arises out of a direct binding of the probe or detection molecule with the transcript or other target molecule. Indirect detection would be a detection of the effects of a gene expression modulation, such as for example, the detection of a protein activity, or a protein expression believed to be connected causally to a modulation in expression of a particular gene. Thus, direct detection means direct hybridization of a detection molecule to a transcript or translation product of a gene.

The term “modulate” as used herein refers to the ability of a molecule to alter the function of another molecule. Thus, modulate could mean, for example, inhibit, antagonize, agonize, upregulate, downregulate, induce, or suppress. A modulator has the capability of altering function of its target. Such alteration can be accomplished at any stage of the transcription, translation, expression or function of the protein, so that, for example, modulation of a target gene can be accomplished by modulation of the DNA, RNA, and protein products of the gene. It assumed that modulation of the function of the target gene will in turn modulate, alter, or affect the function or pathways leading to a function of genes and proteins that would otherwise associate, and interact, or respond to, the target gene. A modulator of the target gene, for example, can be a modulator of an activity of its expressed polypeptide, a modulator of a level of its mRNA transcription, and a modulator of a level of protein expression.

The term “candidate factor” as used herein refers to a compound that is to be tested for its ability to be “a factor that modulates gene expression,” as defined above. Such candidate factors include, for example, expression products of cDNA, genomic DNA, or cRNA libraries derived from any organisms, prokaryotic or eukaryotic. Candidate factor also includes polypeptides, peptides, peptoids, or other small molecules derived from chemical libraries or small molecule libraries. Examples of known small molecule libraries are those disclosed in U.S. Pat. No. 5,010,175, WO 91/17823, WO 91/19735, and patent application U.S. Ser. No. 08/485,006 entitled “Combinatorial Libraries of Substrate-Bound Cyclic Organic Compounds” filed on Jun. 7, 1995, R. Zuckermann et al., J. Am. Chem. Soc. (1992) 114:10646-7, and CHEMISTRY AND BIOCHEMISTRY OF AMINO ACIDS, PEPTIDES, AND PROTEINS—A SURVEY OF RECENT DEVELOPMENTS, Weinstein, B. ed., Marcell Dekker, Inc., publ. New York (1983). This term candidate factors includes factors that are natural products of a producing cell, or products of expression of a producing cell transformed with a polynucleotide encoding such a factor. The candidate factor herein can be secreted, expressed on the surface of a producing cell, or released upon lysis of the producing cell. The candidate factor may be a ligand that binds a receptor where the ligand/receptor complex is capable of triggering an intracellular response in the responsive cell. The candidate factor may be a ligand that is an agonist or antagonist to a receptor. The candidate receptor may be a receptor naturally occurring in a responsive cell or is expressed in a responsive cell upon expression of a polynucleotide encoding the receptor introduced into the responsive cell. The candidate factor may be an extracellular molecule, such as a secreted factor, or an intracellular molecule, such as a transcription factor. Candidate factors may be obtained from serum, tissue, or cell extracts. The factor may also be derived from such sources as plant or animal extracts, or mixtures of extracts from various animal or plant sources. The libraries of candidate factors and candidate receptors can be constructed by use of any of the expression systems practiced by those in the art, or by the methods of synthesis of small molecule libraries known to those skilled in the art.

The term “high throughput” is methodology that permits screening of many candidate factors that modulate gene expression relatively quickly. High throughput generally means that the methodology involves a reduced number of steps or reduced handling of the reagents, such as for example, reduced amounts of washes or transfers of regeants and reactants from one vessel to another. High throughput provides methodology for more candidate factors to be screened in the same time it might have taken to screen less factors by less high throughput and more labor intensive methodology.

A “small amount” as used herein refers to an amount that is reduced or low compared to standard amounts previously used in the same context. In the context of screening for factors that modulate gene expression, a small amount of factor results when a pool of candidate factors is subpooled into smaller amounts, and each subpool therefore only a small amount of molecules of each factor that is being tested in the assay. Where the screening assay takes place in a small reaction vessel, a small amount of candidate factors and a small amount of responding cells are used in the reaction. A great benefit of the invention is that the sensitivity of the reaction permits using small amounts of candidate factors for screening. Thus, the labor, time and expense of making larger amounts of the candidate factors for accomplishing a detection of modulation in a less sensitive assay is not necessary when using the invention. Because small amounts of candidate factors are used, small amounts of responding cells can also be used, and thus detection can be made of the activity of a small amount of molecules of an active factor on the responding cells. The sensitivity of detection allows that increase in transcription, for example, of the amount of responding cells that are placed in a microwell, for example, is possible. Small amount can mean something under 100, a few hundred, or a few thousand molecules or cells, for example, and is a relative term. Generally, a small amount will be a base level amount of a factor that provides a signal detectable by a method of detecting transcription, especially where the detection method is sensitive, for example, by using bDNA detection to detection and amplify the modulation in transcription of transcripts in an amount of cells that can be placed in a microwell and cultured.

An “early intracellular event” as used herein refers to an event that occurs promptly in a responding cell after contact with a factor that modulates gene expression in the cell. The change within the cell may be broader than that indicated by the intracellular event that is detected by the screening assay, although the intracellular event is itself is a modulation. But the intracellular event may be an initial event in a series of events that leads ultimately to a cascade of events that results from a modulation in the expression of one or several genes in the cell. For the purposes of the screening assays of this invention, the intracellular event is a change in the transcription levels of a target gene. The target gene is selected based on the fact that modulation of gene expression of the target gene indicates that the modulating factor will be useful in manipulating the target process being studied or the cellular process for which a therapeutic agent is being sought. Thus, increase in transcript levels of the c-fos gene transcript may indicate, for example, that the modulatory factor causing the increase in transcription is a growth factor or has growth factor activity. The early intracellular event therefore is a change in transcription levels of the gene selected to be detected by the detection means, also known as the target gene. The intracellular event is called early because transcriptional effects are the most immediate of the processes: transcription, translation, protein expression and protein activity.

The term “stimulatory factor” as used herein refers to a subset of “a factor that modulates gene expression” as defined above, that is presently known or unknown, that stimulates transcription, translation, or signal transduction, or otherwise stimulates intracellular activity. Examples of stimulatory factors include growth factors, differentiation factors, factors that stimulate the production of a gene product, such as the ob protein.

The term “inhibitory factor” as used herein refers to a subset of the “factor that modulates gene expression” as defined above, that is presently known or unknown, that inhibits transcription, translation, or signal transduction, or otherwise inhibits intracellular activity.

The term “growth factor” herein refers to a subset of a “stimulatory factor,” defined above, that is presently known or unknown, that stimulates growth of any one or more cell type. Such factors are described generally in Alberts et al., THE MOLECULAR BIOLOGY OF THE CELL (Garland Publishing, NY, N.Y. 1989), and Lewin, GENE V (Oxford Univ. Press, Oxford, England 1994) and include, for example, the family of fibroblast growth factors (FGFs), epidermal growth factor (EGF), platelet derived growth factor (PDGF), insulin-like growth factor (IGF-I and IGF-II), and keratinocyte growth factor (KGF).

The term “differentiation factor” as used herein also refers to a subset of a “stimulatory factor,” that is presently known or unknown, that stimulates the differentiation of one or more cell type, as described generally in Gilbert, DEVELOPMENTAL BIOLOGY (Sinauer Assoc., Sunderland, Mass. 1991). An example of a differentiation factor is nerve growth factor (NGF), or a cytokine.

The term “responsive cell” or “responding cells” as used herein refers to any cell that can respond to the “factor that modulates gene expression” defined above by manifestation of an intracellular event. A responsive cell includes one that can express a receptor or a gene of interest, or that can otherwise manifest an intracellular event such as, for example, signal transduction. Responsive cells appropriate for the invention include both prokaryotic and eukaryotic cells. If eukaryotic, the cells can be mammalian, fungal, insect, avian, worm, fish, crustacean, reptilian, amphibian, or plant cells. The mammalian cells are preferably human cells, but include other animals as well. An example of a responsive cell is the FTL cells derived from NIH3T3 cells. As a further example, where the factor sought is one that stimulates the production of a protein, such as an ob protein as described in Zhang et al. (1994), Nature 372: 425, adipocytes that express ob are one example of responsive cells appropriate herein. Particularly applicable as responsive cells according to the invention are hematopoetic, and neuronal and embryonic stem cells. Where a growth factor is sought, mammalian cells, such as, for example, PC12 cells that express c-fos in response to growth factors can be used as the responsive cells. Some of the mammalian cells that can be responsive cells are, for example, mammalian cell lines including many immortalized cell lines available from the American Type Culture Collection (ATCC), including but not limited to, Chinese hamster ovary (CHO) cells, HeLa cells, baby hamster kidney (BHK) cells, monkey kidney cells (COS), human hepatocellular carcinoma cells (e.g., Hep G2), human embryonic kidney cells, mouse sertoli cells, canine kidney cells, buffalo rat liver cells, human lung cells, human liver cells, mouse mammary tumor cells, as well as others.

The term “producing cell” as used herein refers to a cell that has been enlisted to produce one or more factors or candidate factors. The factors or candidate factors can be a natural product of the producing cell or can be a product of expression of the producing cell transformed with a polynucleotide encoding the factor. Like the responsive cell, the producing cell can be a prokaryotic or an eukaryotic cell as described above. An example of a producing cell is a Xenopus oocyte transfected with a human cRNA library and allowed to express the library.

The term “contacting” as used herein in the context of bringing a factor into close proximity to a responding cell, can be accomplished by conventional means. For example, where the factor is a molecule that can stay in solution, contacting is achieved by adding the factor to the medium containing the responsive cell. “Contacting” herein also includes placing a producing cell in close proximity to a responding cell, where the producing cell either naturally produces a factor or candidate factor or is transformed to produce such factors by introduction of a polynucleotide encoding the factor. In an example, where a library of factors to be screened is injected into one or more Xenopus oocytes for expression, and the responsive cells are mammalian cells such as human cells, contacting the responsive cells can be accomplished by placing one or more oocytes on a bed of responsive cells in a microwell and incubating the cells together at a temperature favoring the mammalian cells. Where the library of candidate factors, whether ligands, stimulatory factors, or inhibitory factors, is a library of polypeptides, peptides, peptoids, or other small molecules, contacting a responsive cell is accomplished by placing one or more of the polypeptides, peptides, peptoids or other small molecules directly in the microwells with the responsive cells.

The term “intracellular event” as used herein refers to an event occurring inside a cell in response to contact with a factor or candidate factor, or in response to ligand/receptor binding. The change includes, for example, an increase or decrease in transcriptional or translational activity in the cell, as well as an increase or decrease in one or more of a chain of events, generally referred to as signal transduction, brought about by the binding of a ligand to a receptor or as a result of cell/cell interaction. For example, an intracellular event can be triggered by the binding of the ligand PDGF to its receptor, the PDGF receptor. The intracellular event includes the phosphorylation of certain proteins or G-protein signaling, leading to activation of certain intracellular pathways.

The term “detecting” as used herein refers to detection of an intracellular event by any appropriate means conventional in the art. The means to detect the intracellular event is tailored to the event. For example, detection of changes in levels of RNA in a cell can be accomplished by bDNA assay, as described in WO 92/02526 or U.S. Pat. No. 5,451,503, and U.S. Pat. No. 4,775,619, or RT-PCR, or RNase protection assay, both as described in Sambrook et al. (1989), MOLECULAR CLONING: A LABORATORY MANUAL, 2d edition (Cold Spring Harbor Press, Cold Spring Harbor, N.Y.), and Ausubel et al., CURRENT PROTOCOLS IN MOLECULAR BIOLOGY (1994), (Greene Publishing Associates and John Wiley & Sons, New York, N.Y.). Where, for example, the invention is used to detect growth factors and the intracellular event is in response to a growth factor stimulation, bDNA specific for c-fos mRNA can be used to detect stimulation of c-fos. Optionally, the c-fos promoter or a regulatory sequence to be modulated can be linked to a reporter gene, and expression of the reporter gene is detected. Methods of detection using reporter genes are known, as described in Sambrook et al. and Ausubel et al. Such reporter genes include, for example, luciferase, chloramphenical acetyl transferase (CAT), green fluorescent protein (GFP), alkaline phosphatase (AP) and β-galactosidase. Where the intracellular event is signal transduction the signal that is transduced is detected by means known in the art, for example, detection of phosphotyrosine with anti-phosphotyrosine antibodies as disclosed in Pasquale et al, “Identification of a developmentally regulated protein-tyrosine kinase by using anti-phosphotyrosine antibodies to screen a cDNA expression library.” Proceedings of the National Academy of Sciences of the United States of America, 1989 July, 86(14):5449-53. Where a differentiation factor is sought, for example, the method of detection can be to observe a phenotypic change of the responsive cells under the microscope, such as, for example, neurite outgrowth of the responsive cell.

The term “a natural product” of a cell as used herein refers to an endogenous product of gene expression in a cell and includes a protein, a polypeptide, or fragments thereof produced by a cell without human intervention of its genetic makeup.

A “natural cell” is a cell not transformed with heterologous DNA, particularly a cell not transformed with a reporter gene.

“Directly detecting” the modulation of a target gene is detection by measuring transcription levels without protein expression, by probe hybridization with a target transcript, and subsequent detection of the hybrid pair formed. The detection is called “direct” because a detection molecule directly binds a transcript. Direct detection provides the opportunity for detection of small amounts of transcripts, where amplification of a probe hybrid can be accomplished from an amplification of the detecting probe molecule, for example, by use of a bDNA detection means.

“Prokaryotic cell” as used herein includes a bacterial and a cyanobacterial cell.

“Eukaryotic cell” as used herein includes a mammalian cell, a fungal cell, an insect cell, an avian cell, a worm cell, a fish cell, a crustacean cell, a reptilian cell, an amphibian cell, and a plant cell, as well as cell lines thereof. An example of an eukaryotic cell as a producing cell is frog Xenopus laevis oocyte.

“Mammalian cell” as used herein refers to a subset of eukaryotic cells and includes human cells, and animal cells such as those from dogs, cats, cattle, horses, rabbits, mice, goats, pigs, etc. The cells used can be genetically unaltered or can be genetically altered, for example, by transformation with appropriate expression vectors, marker genes, and the like. Mammalian cells suitable for the method of the invention are any mammalian cell capable of expressing the genes of interest, or any mammalian cells that can express a cDNA library, cRNA library, genomic DNA library or any protein or polypeptide useful in the method of the invention. Mammalian cells also include cells from cell lines such as those immortalized cell lines available from the American Type Culture Collection (ATCC). Such cell lines include, for example, rat pheochromocytoma cells (PC12 cells), embryonal carcinoma cells (P19 cells), Chinese hamster ovary (CHO) cells, HeLa cells, baby hamster kidney (BHK) cells, monkey kidney cells (COS), human hepatocellular carcinoma cells (e.g., Hep G2), human embryonic kidney cells, mouse sertoli cells, canine kidney cells, buffalo rat liver cells, human lung cells, human liver cells, mouse mammary tumor cells, as well as others. Also included are hematopoetic stem cells, neuronal stem cells such as neuronal sphere cells, and embryonic stem cells (ES cells).

The term “ligand” as used herein refers to a molecule that binds a receptor, such as a protein receptor. A ligand can be a peptide, polypeptide, protein, peptoid or any other molecule capable of forming a binding pair with a receptor. The binding between the ligand and receptor is characterized-as high affinity in order that a binding pair is formed.

The term “receptor” as used herein refers to a molecule such as a protein molecule that binds a ligand to form a binding pair. Receptors are expressed on the cell surface. Binding of a ligand to a receptor transduces a signal through the cell that modulates the cell often by modulateing gene expression in the cell.

The term “antagonist to a receptor” as used herein refers to a ligand that binds a receptor and blocks the binding of other ligands to that receptor but is unable to trigger signal transduction. The antagonist can bind the receptor irreversibly or reversibly.

The term “binding pair” refers to a pair of molecules capable of a binding interaction between the two molecules. Usually a binding interaction furthers a cell signal or cellular event. The term binding pair can refer to a protein/protein, protein-DNA, protein-RNA, DNA-DNA, DNA-RNA, and RNA-RNA binding interactions, and can also include a binding interaction between a small molecule, a peptoid, or a peptide and a protein, DNA, or RNA molecule, in which the components of the pair bind specifically to each other with a higher affinity than to a random molecule, such that upon binding, for example, in case of a ligand/receptor interaction, the binding pair triggers a cellular or an intracellular response. An example of a ligand/receptor binding pair is a pair formed between PDGF (platelet derived growth factor) and a PDGF receptor. An example of a different binding pair is an antigen/antibody pair in which the antibody is generated by immunization of a host with the antigen. Another example of a binding pair is the formation of a binding pair between a protease and a protease inhibitor, or a protease substrate and a protease inhibitor. Specific binding indicates a binding interaction having a low dissociation constant, which distinguishes specific binding from non-specific, background, binding.

A “nucleic acid molecule” or a “polynucleotide,” as used herein, refers to either RNA or DNA molecule that encodes a specific amino acid sequence or its complementary strand. Nucleic acid molecules may also be n example, a ribozyme, an antisense oligonucleotide, or an untra A “coding sequence” as used herein, refers to either RNA or D amino acid sequence or its complementary strand. A polynucl example, an antisense oligonucleotide, or a ribozyme, and may also include such items as a 3′ or 5′ untranslated region of a gene, or an intron of a gene, or other region of a gene that does not make up the coding region of the gene. The DNA or RNA may be single stranded or double stranded. Synthetic nucleic acids or synthetic polynucleotides can be chemically synthesized nucleic acid sequences, and may also be modified with chemical moieties to render the molecule resistant to degradation. Synthetic nucleic acids can be ribozymes or antisense molecules, for example. Modifications to synthetic nucleic acid molecules include nucleic acid monomers or derivative or modifications thereof, including chemical moieties. For example, phosphothioates can be used for the modification. A polynucleotide derivative can include, for example, such polynucleotides as branched DNA (bDNA). A polynucleotide can be a synthetic or recombinant polynucleotide, and can be generated, for example, by polymerase chain reaction (PCR) amplification, or recombinant expression of complementary DNA or RNA, or by chemical synthesis.

A “regulatory sequence”, herein refers to a nucleic acid sequence encoding one or more elements that are capable of modulateing or effecting expression of a gene sequence, including transcription or translation thereof, when the gene sequence is placed in such a position as to subject it to the control thereof. Such a regulatory sequence can be, for example, a minimal promoter sequence, a complete promoter sequence, an induced active promoter, an enhancer sequence, an upstream activation sequence (“UAS”), an operator sequence, a downstream termination sequence, a polyadenylation sequence, an optimal 5′ leader sequence to optimize initiation of translation, or a Shine-Dalgamo sequence. Alternatively, the regulatory sequence can contain a hybrid of promoters of any of the above, such as a hybrid enhancer/promoter element. The regulatory sequence that is appropriate for expression of the gene of interest differs depending upon the host system in which the construct is to be expressed. Selection of the appropriate regulatory sequences for use herein is within the capability of one skilled in the art. In eukaryotes, for example, such a sequence can include one or more of a promoter sequence and/or a transcription termination sequence. Regulatory sequences suitable for use herein may be derived from any source including a prokaryotic source, an eukaryotic source, a virus, a viral vector, a bacteriophage or a linear or circular plasmid. The regulatory sequence herein can also be a synthetic sequence, for example, one made by combining the UAS of one gene with the remainder of a requisite promoter from another gene, such as the GADP/ADH2 hybrid promoter. A regulatory sequence can also be a repressor sequence.

The term “tissue marker” as used herein refers to any protein or fragments thereof expressed in a cell or tissue that, by virtue of the expression of that protein or fragment, identifies the cell or tissue. Tissue markers are useful herein for detecting or identifying differentiation factors. Where a differentiation factor is sought from a pool of candidate factors, the responsive cells will be assayed for increased transcription or translation of a tissue marker specific for the cells used for the assay. Although, to practice the invention, the tissue marker or gene to be assayed need not be known, where there exist known tissue markers, these can be used for screening for differentiation factors that are responsible for the differentiation. Some tissue markers that can be used for this purpose include genes expressed in a tissue specific or cell specific manner, including both known and unknown genes. The invention includes a means for detecting unknown genes by use of mRNA differential display. Tissue markers that can be used for screening for differentiation factors include, but are not limited to, for example, neuron-specific enolase, as described in Forss-Petter et al. (1990) Neuron 5:187-197; insulin; inducible nitric oxide synthase; interferon regulatory factor 1; interferon regulatory factor 2; interferonrstimulated gene factor-3γ (ISGFR3γ); brachyury; goosecoid: muscle actin; and IVCAM. Other tissue markers can be protein hormones, cytokines, cell adhesion molecules, proteases, serum binding proteins, enzymes such as hydroxylases, neuron specific proteins, cell surface receptors, and proteins specific to the immune system.

“Markers” for transcriptional events that modulate gene expression can include changes in transcript levels of any gene the transcription of which is modulated as a result of a modulatory factor. Such markers can include, but are not limited to, for example, immediate early genes, cytokines, transcription factors, protein hornones, signaling molecules, apoptotic genes, oncogenes, protooncogenes, tumor suppressor genes, genes associated with inflammation, hematopoetic genes, genes associated with neuronal signalling and activity, ahd in general any gene known or believed to be induced by another gene's activity. Additionally, neuron-inducer factors including neuron-specific enolase, insulin inducer factors, interfereon regulatory factor I, interferon regulatory factor 2, interferon-stimulated gene factor-3 gamma, mesoderm inducers such as Brachyury and goosecoid, and neuronal tissue inducer such as muscle actin and IV CAM. Some specific examples of such markers include, for example, IL-2, IL-6, NFKB elements, A20 (an apoptotic gene), inducible nitric oxide synthetase, c-fos, c-myc, interferon, beta globulin, peripherin, interferon inducible elements including p48, IRF-1, CIS, and OSM. Other potential markers are described, for example, in Faisst and Meyer, Nucleic Acids Research, 20 (1); 3-26 (1992), herein incorporated by reference in full. Still further genes that can act as markers for use in the assay are listed in Darnell et al., MOLECULAR CELL BIOLOGY, Scientific American Books, NY, 1990, page 408, incorporated by reference in full. Another example of gene markers for transcriptional events includes those described in Dhawale and Lane, Nucleic Acids Research, v. 21(24): 5537-5546 (1993), also incorporated by reference in full. Still other examples of possible markers include those described in Wingender, Nucleic Acids Res. 16(5) 1880-1992 (1988), Bustin and McKay, Brit J. Biomed. Sci. 51:147-157 (1994), Peterson and Tupy, Biochem. Pharm. 47: 127-128 (1994), and Ghosh, Nucleic Acids Research, 20 supp: 2091-2093 (1993), all incorporated by reference in full.

The term “protein” or “polypeptide” used herein in the context of a factor or product of a gene expressed or regulated includes “mature protein” and “analogs” thereof that are truncations, variants, allelles and derivatives of the mature protein. Unless specifically mentioned otherwise, the “analogs” possess one or more of the bioactivities of the “mature protein.” Thus, polypeptides that are identical or contain at least 60%, preferably 70%, more preferably 80%, and most preferably 90% amino acid sequence homology to the amino acid sequence of the mature protein wherever derived, from human or nonhuman sources, are included within this definition.

The “variants” herein contain amino acid substitutions, deletions, or insertions. The amino acid substitutions can be conservative amino acid substitutions or substitutions to eliminate non-essential amino acid residues such as to alter a glycosylation site, a phosphorylation site, an acetylation site, or to minimize misfolding by substitution or deletion of one or more cysteine residues that are not necessary for function. Conservative amino acid substitutions are those that preserve the general charge, hydrophobicity/hydrophilicity and/or steric bulk of the amino acid substituted, for example, substitutions between the members of the following groups are conservative substitutions: Gly/Ala, Val/Ile/Leu, Asp/Glu, Lys/Arg, Asn/Gln, Ser/Cys/Thr and Phe/Trp/Tyr. The analogs herein further include peptides having one or more peptide mimics, also known as peptoids, that possess the bioactivity of the protein. Included within the definition are also polypeptides containing one or more analog amino acid (including, for example, unnatural amino acids, etc.), polypeptides with substituted linkages, as well as other modifications known in the art, both naturally occurring and non-naturally occurring. The term polypeptide also does not exclude post-expression modifications of the polypeptide, for example, glycosylations, acetylations, phosphorylations and the like.

The term “co-culturing” as used herein refers to the state of culturing more than one type of cell together. For example, co-culturing in the screening assay can be with producing cells and responding cells, where the producing cells would be different from the responding cells primarily because they would express and secrete proteins from cDNA or cRNA that had been introduced into the producing cell. Co-culturing can also take place between, for example, a Xenopus cell and a mammalian cell, or two different mammalian cells, or other cells of the same species or different species. A unique feature of the invention is the achievement of the ability to co-culture Xenopus oocytes with mammalian cells, the methodology of which is described later, but which involves temperature control and control of the co-culture time periods. The usefulness of co-culturing cells in this invention is the ability of a producing cells to express and secrete factors that can act upon the responding cells which are cultured in the same media.

The word “discovered” as used herein is identification of a factor capable of modulating the expression of a gene where that factor was not previously known as a factor that could facilitate or cause that modulation. For example, a factor that is discovered as a growth factor, may be a previously known factor, but the discovery is that the factor can modulate growth, a fact that was previously unknown.

In one embodiment of the present invention, a method is utilized to detect or identify or screen a novel factor, such as a growth factor. This can be done by creating contact between a candidate factor or factors to be tested with a population of responsive cells, and looking for a response in the cells to such contact. The candidate factor can be either added to the medium containing the responsive cells, as in the case of a small molecule library, or can be a product of expression of one or more producing cells. The producing cells may be one that produces the candidate factor naturally or is transformed with a polynucleotide, such as DNA or RNA, encoding the candidate factor.

Thus, the producing cell can be made to produce the candidate factor by introducing therein the polynucleotide. The polynucleotide can be introduced into the producing cell by any conventional methods including, for example, electroporation, calcium phosphate treatment, and transfection, such as lipofectamine transfection. In one embodiment of the present invention, the polynucleotide is inserted into a vector or a plasmid suitable for expression of the polynucleotide in a producing cell. The vector or plasmid can be one that is capable of independent replication or can be one that is capable of integration into the host genome.

The polynucleotide encoding the candidate factor can be a known fragment isolatable from a known source or can be derived from a pool of polynucleotides derived from for example, plasmids containing a cDNA library, cRNA library, or genomic DNA library. Conventional linkers or polylinkers can be used in constructing such vectors containing the polynucleotide that encodes the candidate factor.

One or more producing cells may be used in the present invention. As an example, a Xenopus laevis oocyte may be used as a producing cell herein. The type of cells to be selected as producing cells depends upon the factor to be identified or screened. For example, if the candidate factor resides in an eukaryotic cell DNA library, the producing cells preferably are eukaryotic cells. If the producing cells are transformed to produce the candidate factor, a stable cell line containing the candidate factor encoding polynucleotide is preferably first obtained and progenies of such cell line are used in the present method. Alternatively, one or more producing cells can be transformed with plasmids containing a polynucleotide library, and the producing cells can be used in the present method and allowed to express such library.

The producing cells or the responding or responsive cells can be cells derived from any organism, including, for example, a mammalian cell, a fungal cell, including a yeast cell, an insect cell, an avian cell, a worm cell, a fish cell, a crustacean cell, a reptilian cell, an amphibian cell, a bacterial cell, and a plant cell. The genes uses as a readout of the gene expression modulation by the factor sought can be gene also derived from any organism, including, for example from a mammal, a fungi, including a yeast, an insect, a bird, a worm, a fish, a crustacean, an amphibian, a reptile, a bacterium, and a plant.

The responding cells or responsive cells herein are also selected on the basis of the factor to be screened or identified. For example, if a human growth factor is to be detected, the responsive cells preferably are human cells. If the responsive cells are to be transformed, for example, to produce a candidate receptor, a stable cell line containing the candidate receptor may be selected and its progenies used in the present method. Alternatively, a population of responsive cells can be transformed with for example, plasmids containing a polynucleotide library, and such population of responsive cells can be used in the present method and the cells are allowed to express such library.

The requisite contact between the producing cells or candidate factors and the responsive cells herein is achieved by culturing the responsive cells in the presence of the factor or producing cells at a temperature suitable for survival of the responsive cells and for transcription, translation, signal transduction or other intracellular activity to take place. Thus, serum, tissue, or cell extracts may also be used in such a culture if such serum, tissue, or cell extract is the source of the candidate. The cells can be incubated in a suitable container or dish or preferably, a micro well in a microtiter plate for a suitable period of time for the intracellular event to take place.

The intracellular event or response in the responsive cells to the candidate factor may be detected by any appropriate means sufficiently sensitive to detect such a response preferably, a bDNA assay for detecting transcription or translation activity, and an antibody assay for detecting phosphorylation. Detection can also be accomplished by use of RT-PCR or RNase protection assay. Generally detection of change in transcript levels can be used for as a detection of an early intracellular event, because transcriptional changes occur before translation, expression, or protein activity for a given gene. Detection of transcriptional changes also provides the most sensitive detection possible due to the ability of transcriptional detection systems to provide a signal with very little change in transcript levels.

The detection means will be targeted to a gene of interest, the modulation of which is significant. So that, for example, where a differentiation factor is sought, for example, a gene associated with differentiation is the target gene, where an inhibitory factor is sought, a gene associated with the inhibition of growth of a cell is sought, and where a ligand to a receptor is sought, a gene associated with an increased activity believed to be caused by increased receptor activity is sought. Examples of some of these genes that could be used as a readout for detecting the factors sought by detecting increase or decrease in the levels of transcription of the gene, for example, are described in MOLECULAR AND CELL BIOLOGY, Darnell et al Ed., Scientific American Books, N.Y. 1990, Faisst and Meyer, Nucleic Acids Research, v. 20 (1) 3-26 (1992), Dhawale and Lane, Nucleic Acids Research, v. 21(24) 5537-5546 (1993).

Where a receptor is sought, the candidate receptor molecules can be expressed from cDNA, cRNA or genomic DNA libraries in either the producing cells or the responsive cells, preferably on the surface thereof. If the candidate receptor molecules are expressed on producing cells, the responsive cell may also expressed receptor molecules that interact with the receptor molecules on the producing cells, such interaction triggering an intracellular event in the responsive cells. If the candidate receptor is expressed on the responsive cells, the producing cells may express a ligand that interacts with the receptor molecules, triggering an intracellular event in the responsive cells.

Where a transcription factor is sought, as with the screening for a receptor, the producing cell and the responsive cell are the same cell, and the transcription factor is sought from among the non-secreted factors produced from the library that is screened.

The intracellular event can be increase or decrease of transcription of a gene, or can be a signal transduction event such as, for example, phosphorylation or G-protein signaling.

Where the gene for which increase or decrease of transcription in response to the sought-after factor is known, detection of a change in transcription of a gene is accomplished, for example, by bDNA technology such as that disclosed in U.S. Pat. No. 5,451,503 and WO 92/02526. The bDNA selected is to that gene expressed in the responding cells for which increase or decrease of transcription is expected. So that in the case where a growth factor is sought, bDNA to the c-fos transcript is used to detect an increase in c-fos gene transcription. Also for example, where an inhibitor is sought, bDNA to the IL-2 transcript can be used to detect a decrease in IL-2 transcription in responding cells that express IL-2. Where, for example, a ligand is sought, responsive cells that respond to the ligand/receptor binding pair with up- or down-regulation of transcription of a gene can be used, and bDNA to that gene transcript can be used to detect the positive. Where, for example, an antagonist to a receptor is sought, responsive cells will respond to the antagonist/receptor binding pair with down-regulation of a gene normally up-regulated by the ligand/receptor binding pair, and this decrease in transcription is detected by bDNA to the regulated gene.

Where, for example, a transcription factor that acts at a regulatory sequence is sought, the regulatory sequence is linked to a reporter gene, or to the gene the regulatory sequence normally regulates, and bDNA to the reporter gene or to the native gene is used to detect the presence of the transcription factor in particular pool of candidates. Reporter genes that can be used for this purpose include, but are not limited to, luciferase, βGalactosidase (βGal), cholamphenicol acetyl transferase (CAT), green fluorescent protein (GFP), and secreted alkaline phosphatase (SEAP). Other reporter genes appropriate for this function are known and used in the art.

Where, for example, a receptor to a ligand is sought, bDNA to the gene the binding pair regulates is used to detect the presence of the receptor on the surface of the producing/responding cell, as described for detection of a ligand.

Other means for detection of an intracellular event where the event is increase or decrease of transcription of a gene include RNase protection assay and RT-PCR, both as described in Sambrook et al. and Ausubel et al., cited previously. These protocols are applied to detect an increase or decrease in the transcription of a gene for the situation where stimulatory factors, inhibitory factors, ligands, receptors, antagonists to receptors and transcription factors are sought, in basically the same manner as described for the bDNA assays above.

A special circumstance for use of RT-PCR applies to the invention, where the gene that will be up- or down-regulated is not known. The process is called mRNA differential display system. The process is disclosed in U.S. Pat. No. 5,262,311 and Liang et al. (1992) Science 257:967-971, and is produced by GenHunter Corporation under the trade name RNAimage™. An example of how this technique is useful in the invention is where responsive cells respond to the serum by increase growth. These responsive cells are incubated with the serum, and RT-PCR using random primers is used to identify by differential display of RT-PCR products, the gene that is up-regulated by the serum. That gene is sequenced from the RT-PCR product and bDNA probes made for that gene from the sequence information. The invention then proceeds as previously described: the serum is sub-divided, responsive cells cultured in the presence of the serum pools, positives are detected by bDNA assay (or RNase protection assay or RT-PCR), and the growth factor eventually isolated.

Where a receptor is sought, or a ligand is sought, or an antagonist to a receptor is sought, and the intracellular event is signal transduction, an assay to detect the signal transduction is used to detect the ligand/receptor binding pair, or the antagonist/receptor binding pair. Such methods to detect the signal transduction include detection of the phosphorylation of intermediates in the signal transduction as disclosed in Pasquale et al, “Identification of a developmentally regulated protein-tyrosine kinase by using anti-phosphotyrosine antibodies to screen a cDNA expression library.” Proceedings of the National Academy of Sciences of the United States of America, 1989 July, 86(14):5449-53.

The invention can also be used in a context where a patient's primary cells are removed and screened by the assay for effectiveness of use of a proposed therapeutic agent, for example. Thus, the patients cells from tissue of an affected organ, or tumor tissue, for example, could be removed, cultured in the presence of different therapeutic agents, and the desired modulatory effect screened for by, for example, detection with branched DNA or RT-PCR of a target gene transcript. This approach might facilitate quick assessment of an appropriate therapeutic agent for a given individual patient, in advance of an actual administration of an agent. Because the screening assay is quick, little time would be lost in starting effective treatment for the patient. This approach might also be used as a secondary screen to test a proposed therapeutic agent for efficacy in with the cells of a population of patients, thus, providing a secondary, but pre-clinical assay to indicate the likelihood of success of a particular therapeutic agent.

To practice the invention, a source of the candidate stimulatory factors, inhibitory factors, candidate receptors, candidate ligands, candidate transcription factors, or candidate antagonists to a receptor is first chosen. The assay can be conducted, for example in plates of 96 microwells each. The source of the candidates is divided into pools, so that, from 100 to 1000 producing cells make up each pool. Responsive cells are selected that are appropriate for the candidates being screened and that can express the gene to be identified or detected.

In the special case where a receptor is sought, the responsive cells are also the producing cells, and both express the sought-after receptor, and respond to the ligand/receptor binding pair. Receptors that can be sought by the method of the invention include receptors to ligands such as Noggin, Wnt and Notch.

In the case where the factor that is sought acts at a response element or acts to stimulate factors that act at response elements, constructs containing the regulatory sequence of the gene of interest linked to either the gene that the regulatory element normally regulates, or a reporter gene, are stably transfected into appropriate responsive cells that can express the gene under the control of the regulatory sequence. This cell line becomes the source of the responsive cells. To assay for transcription factors that up-or down-regulate at the regulatory element, the responsive cells are incubated with a library of candidate factors, such as, for example, a library expressed and secreted by producing cells transformed by the library. Detection of the change in transcription is accomplished by, for example, the reporter gene detection assay, bDNA to the reporter gene, or bDNA to the gene under regulatory control of the regulatory element. Other exemplary response elements that can be assayed by the method of this invention to find factors that activate response elements and for which there is presently limited knowledge about the transcription factors that act at these response elements are NFKB responsive element, interferon responsive element or pIRE, interferon-stimulated response enhancers or ISRE, interferon-γ-activated sequences or GAS, and regulatory sequences including promoters, enhancers and repressors for, the following regulated genes, neuron-specific enolase, insulin, inducible nitric oxide synthase, interferon regulatory factor 1, interferon regulatory factor 2, interferon-stimulated gene factor-3γ (ISGFR3γ), brachyury, goosecoid, muscle actin, cell adhesion molecules-4 or IVCAM. In general, any regulatory element can be assayed by this invention to identify the factors that control them.

Stimulators and inhibitors of gene transcription, ligands, and antagonists to receptors can be sought from small molecule libraries. Responsive cells are selected that express a gene sought to be stimulated or inhibited, or responsive cells that can manifest a signal transduction in response to a ligand/receptor binding pair interaction on its cell surface are selected. The responsive cells are incubated in the presence of pools of small molecules. Positives are subdivided until small molecule stimulator, inhibitor, ligand or antagonist is identified.

The invention can be used to look for stimulatory or inhibitory factors of any gene. Some of the genes for which a stimulator or inhibitor might be sought, and could be sought by the method of invention, include, for example, extracellular molecules such as, for example, protein hormones, cytokines, lymphokines, growth factors, differention factors, extracellular matrix molecules, and intracellular molecules signalling molecules including transcription factors and cell surface receptors or nuclear receptors. When an extracellular factor is sought, secreted factors are screened, and when an intracellular molecule is sought, the producing cell and the responsive cell are one in the same cell. Specific examples of some of these categories include the gene products IL-2, c/EBP alpha, cyclin D, ob protein, A20 protein, cell adhesion molecule-1 (ICAM-1), and gene products of other proteins that are induced by cytokines like TNF, IL-2, IL-3, IL-6, IL-8; c-fos and other proliferation markers, proteins that are induced by growth factors like PDGF, EGF, and KGF, and differentiation factors like neuronal growth factor (NGF).

Genes the expression of which can be used in the screening assay for detecting a factor that modulates a gene's expression can be a gene encoding a DNA-binding protein, a disease marker, a growth marker, a differentiation marker, an apoptotic marker, a metastatic marker, a marker associated with a later onset of a disease, and an oncogene.

For all the genes for which modulatory factors are sought, the gene of interest can be used as a target for a detection system for the factor, or another gene, the modulation of expression of which is associated with the modulation of the gene of interest, can also be used.

The following sections describe exemplary methods of expression that can be employed in the invention, cells that can be either producing cells or responsive cells in the invention, and ways that libraries of cDNA, cRNA or genomic DNA, or small molecules can be constructed to provide sources of growth, differentiation, transcription, or inhibitory factors, ligands, receptors, or receptor antagonists. cDNA libraries of candidate factors can be generated from any genome desired. Poly A+ mRNA is isolated from the selected cells or tissue, and cDNA made from the RNA using reverse transcriptase enzyme. The pool of cDNAs generated is then ligated into vectors which can be transformed into the cells appropriate for the vector, which include for example, the cells listed herein and particularly bacterial cells. A person skilled in the art would be able to select such a vector and host cell for such purposes. The pools are created, for example, from 100 to 1000 colonies of cells per pool. Examples of suitable vectors and host cells are disclosed below. Vectors containing the cDNA are introduced into host cells such as, for example, those cells listed herein, by conventional techniques including electroporation, calcium phosphate treatment, lipofectamine transfection, and microinjection.

The genes or target genes the modulation of transcription of which can be detected by the detection means can be any gene for which the modulation of the expression of which is an indicator that a factor is a modulating factor in the manner sought. Thus, for example, where a factor capable of modulating gene expression that results in cell growth is sought, the target gene will be that gene that demonstrates an increased level of transcription prior to or coincident with cell growth. So that, for example, such a gene that encodes a DNA-binding protein, a disease marker, a growth marker, a differentiation marker, an apoptotic marker, a metastatic marker, a marker associated with a later onset of a disease, and an oncogene, can be a target gene for the purposes of the invention, and the transcription levels of this gene can be detected by the invention for the purpose of identifying a factor capable of modulating the expression of a gene of interest. The gene of interest may be the target gene, or may be a gene the modulation of expression of which is associated with or caused by the modulation of expression of the target gene. Also, by example, the target gene, the modulation of which is detected by the invention can be a gene encoding a leptin protein, and A20 protein, ICAM, c-fos protein, c-myc protein, and others.

Once a pool is identified to contain a positive factor, that pool is subdivided into, for example 10 sub-pools, and rescreened. The positive pool from these subpools is subdivided into, for example 10 sub-subpools, and so on, until the positive pool produces one single colony, representing the clone of the factor that generates the positive response. This clone is under the regulatory control of the vector of the plasmid, and can be sequenced from this vector using the primers complementary to the 5′ and 3′ ends of the vector linked to the cDNA of the factor. The clone may also be isolated from the vector and placed into an expression cassette for large scale production of the polypeptide encoding the factor in the appropriate host cells. The vectors and host cells and expression cassette are selected from those materials available in the art. Once the factor has been isolated and the cDNA sequenced, the full length gene can be sought from the genomic DNA encoding the factor and its regulatory sequences.

Expression Systems

Although the methodology described below is believed to contain sufficient details to enable one skilled in the art to practice the present invention, other items not specifically exemplified, such as plasmids, can be constructed and purified using standard recombinant DNA techniques described in, for example, Sambrook et al. (1989), MOLECULAR CLONING: A LABORATORY MANUAL, 2d edition (Cold Spring Harbor Press, Cold Spring Harbor, N.Y.), and Ausubel et al., CURRENT PROTOCOLS IN MOLECULAR BIOLOGY (1994), (Greene Publishing Associates and John Wiley & Sons, New York, N.Y.). under the current regulations described in United States Dept. of HEW, NATIONAL INSTITUTE OF HEALTH (NIH) GUIDELINES FOR RECOMBINANT DNA RESEARCH. These references include procedures for the following standard methods: cloning procedures with plasmids, transformation of host cells, cell culture, plasmid DNA purification, phenol extraction of DNA, ethanol precipitation of DNA, agarose gel electrophoresis, purification of DNA fragments from agarose gels, and restriction endonuclease and other DNA-modifying enzyme reactions.

Where a ligand must be constructed to activate a candidate receptor expressed on the surface of a responsive cell, standard methods of expression can be used as described as follows for bacterial, yeast, insect and mammalian expression systems, and these expression systems can either be used as producing cells to express ligand which is then purified, or to express non-diffusible ligand to contact the responsive cells. The following expression systems are also applicable to the construction of cDNA or cRNA libraries, where secreted proteins are generated for screening for their ability to alter gene expression in responsive cells. The expression systems are also applicable to the construction of regulatory elements linked with reporter genes for identification of factors which stimulate or inhibit by acting at the regulatory elements. The following cells are appropriate as both responsive cells or as the cells from which libraries of candidate factors are generated, also called producing cells.

Expression in Bacterial Cells

Control elements for use in bacteria include promoters, optionally containing operator sequences, and ribosome binding sites. Useful promoters include sequences derived from sugar metabolizing enzymes, such as galactose, lactose (lac) and maltose. Additional examples include promoter sequences derived from biosynthetic enzymes such as tryptophan (trp), the β-lactamase (bla) promoter system, bacteriophage λPL, and T7. In addition, synthetic promoters can be-used, such as the tac promoter. The β-lactamase and lactose promoter systems are described in Chang et al., Nature (1978) 275: 615, and Goeddel et al., Nature (1979) 281: 544; the alkaline phosphatase, tryptophan (trp) promoter system are described in Goeddel et al., Nucleic Acids Res. (1980) 8: 4057 and EP 36,776 and hybrid promoters such as the tac promoter is described in U.S. Pat. No. 4,551,433 and de Boer et al., Proc. Natl. Acad. Sci. USA (1983) 80: 21-25. However, other known bacterial promoters useful for expression of eukaryotic proteins are also suitable. A person skilled in the art would be able to operably ligate such promoters to the coding sequences of interest, for example, as described in Siebenlist et al., Cell (1980) 20: 269, using linkers or adaptors to supply any required restriction sites. Promoters for use in bacterial systems also generally will contain a Shine-Dalgarno (SD) sequence operably linked to the DNA encoding the target polypeptide. For prokaryotic host cells that do not recognize and process the native target polypeptide signal sequence, the signal sequence can be substituted by a prokaryotic signal sequence selected, for example, from the group of the alkaline phosphatase, penicillinase, Ipp, or heat stable enterotoxin II leaders. The origin of replication from the plasmid pBR322 is suitable for most Gram-negative bacteria.

The foregoing systems are particularly compatible with Escherichia coli. However, numerous other systems for use in bacterial hosts including Gram-negative or Gram-positive organisms such as Bacillus spp., Streptococcus spp., Streptomyces spp., Pseudomonas species such as P. aeruginosa, Salmonella typhimurium, or Serratia marcescans, among others. Methods for introducing exogenous DNA into these hosts typically include the use of CaCl₂ or other agents, such as divalent cations and DMSO. DNA can also be introduced into bacterial cells by electroporation, nuclear injection, or protoplast fusion as described generally in Sambrook et al. (1989), cited above. These examples are illustrative rather than limiting. Preferably, the host cell should secrete minimal amounts of proteolytic enzymes. Alternatively, in vitro methods of cloning, e.g., PCR or other nucleic acid polymerase reactions, are suitable.

Prokaryotic cells used to produce the target polypeptide of this invention are cultured in suitable media, as described generally in Sambrook et al., cited above.

Expression in Yeast Cells

Expression and transformation vectors, either extrachromosomal replicons or integrating vectors, have been developed for transformation into many yeasts. For example, expression vectors have been developed for, among others, the following yeasts: Saccharomyces cerevisiae, as described in Hinnen et al., Proc. Natl. Acad. Sci. USA (1978) 75: 1929; Ito et al., J. Bacteriol. (1983) 153: 163; Candida albicans as described in Kurtz et al., Mol. Cell. Biol. (1986) 6: 142; Candida maltosa, as described in Kunze et al., J. Basic Microbiol. (1985) 25: 141; Hansenula polymorpha, as described in Gleeson et al., J. Gen. Microbiol. (1986) 132: 3459 and Roggenkamp et al., Mol. Gen. Genet. (1986) 202:302); Kluyveromyces fragilis, as described in Das et al., J. Bacteriol. (1984) 158: 1165; Kluyveromyces lactis, as described in De Louvencourt et al., J. Bacteriol. (1983) 154: 737 and Van den Berg et al., Bio/Technology (1990) 8: 135; Pichia guillerimondii, as described in Kunze et al., J. Basic Microbiol. (1985) 25: 141; Pichia pastoris, as described in Cregg et al., Mol. Cell. Biol. (1985) 5: 3376 and U.S. Pat. Nos. 4,837,148 and 4,929,555; Schizosaccharomyces pombe, as described in Beach and Nurse, Nature (1981) 300: 706; and Yarrowia lipolytica, as described in Davidow et al., Curr. Genet. (1985) 10: 380 and Gaillardin et al., Curr. Genet. (1985) 10: 49, Aspergillus hosts such as A. nidulans, as described in Ballance et al., Biochem. Biophys. Res. Commun. (1983) 112: 284-289; Tilburn et al., Gene (1983) 26: 205-221 and Yelton et al., Proc. Natl. Acad. Sci. USA (1984) 81: 1470-1474, and A. niger, as described in Kelly and Hynes, EMBO J. (1985) 4: 475479; Trichoderna reesia, as described in EP 244,234, and filamentous fungi such as, e.g, Neurospora, Penicillium, Tolypocladium, as described in WO 91/00357.

Control sequences for yeast vectors are known and include promoters regions from genes such as alcohol dehydrogenase (ADH), as described in EP 284,044, enolase, glucokinase, glucose-6-phosphate isomerase, glyceraldehyde-3-phosphate-dehydrogenase (GAP or GAPDH), hexokinase, phosphofructokinase, 3-phosphoglycerate mutase, and pyruvate kinase (PyK), as described in EP 329,203. The yeast PHO5 gene, encoding acid phosphatase, also provides useful promoter sequences, as described in Myanohara et al., Proc. Natl. Acad. Sci. USA (1983) 80: 1. Other suitable promoter sequences for use with yeast hosts include the promoters for 3-phosphoglycerate kinase, as described in Hitzeman et al., J. Biol. Chem. (1980) 255: 2073, or other glycolytic enzymes, such as pyruvate decarboxylase, triosephosphate isomerase, and phosphoglucose isomerase, as described in Hess et al., J. Adv. Enzyme Reg. (1968) 7: 149 and Holland et al., Biochemistry (1978) 17:4900. Inducible yeast promoters having the additional advantage of transcription controlled by growth conditions, include from the list above and others the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in Hitzeman, EP 073,657. Yeast enhancers also are advantageously used with yeast promoters. In addition, synthetic promoters which do not occur in nature also function as yeast promoters. For example, upstream activating sequences (UAS) of one yeast promoter may be joined with the transcription activation region of another yeast promoter, creating a synthetic hybrid promoter. Examples of such hybrid promoters include the ADH regulatory sequence linked to the GAP transcription activation region, as described in U.S. Pat. Nos. 4,876,197 and 4,880,734. Other examples of hybrid promoters include promoters which consist of the regulatory sequences of either the ADH2, GAL4, GAL10, or PHO5 genes, combined with the transcriptional activation region of a glycolytic enzyme gene such as GAP or PyK, as described in EP 164,556. Furthermore, a yeast promoter can include naturally occurring promoters of non-yeast origin that have the ability to bind yeast RNA polymerase and initiate transcription.

Other control elements which may be included in the yeast expression vectors are terminators, for example, from GAPDH and from the enolase gene, as described in Holland et al., J. Biol. Chem. (1981) 256: 1385, and leader sequences which encode signal sequences for secretion. DNA encoding suitable signal sequences can be derived from genes for secreted yeast proteins, such as the yeast invertase gene as described in EP 012,873 and JP 62,096,086 and the α-factor gene, as described in U.S. Pat. Nos. 4,588,684, 4,546,083 and 4,870,008; EP 324,274; and WO 89/02463. Alternatively, leaders of non-yeast origin, such as an interferon leader, also provide for secretion in yeast, as described in EP 060,057.

Methods of introducing exogenous DNA into yeast hosts are well known in the art, and typically include either the transformation of spheroplasts or of intact yeast cells treated with alkali cations.

Transformations into yeast can be carried out according to the method described in Van Solingen et al., J. Bact. (1977) 130:946 and Hsiao et al., Proc. Natl. Acad. Sci. (USA) (1979) 76:3829. However, other methods for introducing DNA into cells such as by nuclear injection, electroporation, or protoplast fusion may also be used as described generally in Sambrook et al., cited above.

For yeast secretion the native target polypeptide signal sequence may be substituted by the yeast invertase, α-factor, or acid phosphatase leaders. The origin of replication from the 2μ plasmid origin is suitable for yeast. A suitable selection gene for use in yeast is the trp1 gene present in the yeast plasmid described in Kingsman et al., Gene (1979) 7: 141 or Tschemper et al., Gene (1980) 10:157. The trp1 gene provides a selection marker for a mutant strain of yeast lacking the ability to grow in tryptophan. Similarly, Leu2-deficient yeast strains (ATCC 20,622 or 38,626) are complemented by known plasmids bearing the Leu2 Gene.

For intracellular production of the present polypeptides in yeast, a sequence encoding a yeast protein can be linked to a coding sequence of the polypeptide to produce a fusion protein that can be cleaved intracellularly by the yeast cells upon expression. An example, of such a yeast leader sequence is the yeast ubiquitin gene.

Expression in Insect Cells

Baculovirus expression vectors (BEVs) are recombinant insect viruses in which the coding sequence for a foreign gene to be expressed is inserted behind a baculovirus promoter in place of a viral gene, e.g., polyhedrin, as described in Smith and Summers, U.S. Pat. No., 4,745,051.

An expression construct herein includes a DNA vector useful as an intermediate for the infection or transformation of an insect cell system, the vector generally containing DNA coding for a baculovirus transcriptional promoter, optionally but preferably, followed downstream by an insect signal DNA sequence capable of directing secretion of a desired protein, and a site for insertion of the foreign gene encoding the foreign protein, the signal DNA sequence and the foreign gene being placed under the transcriptional control of a baculovirus promoter, the foreign gene herein being the coding sequence of the polypeptide.

The promoter for use herein can be a baculovirus transcriptional promoter region derived from any of the over 500 baculoviruses generally infecting insects, such as, for example, the Orders Lepidoptera, Diptera, Orthoptera, Coleoptera and Hymenoptera including, for example, but not limited to the viral DNAs of Autographo californica MNPV, Bombyx mori NPV, rrichoplusia ni MNPV, Rachiplusia ou MNPV or Galleria mellonella MNPV. Thus, the baculovirus transcriptional promoter can be, for example, a baculovirus immediate-early gene IEI or IEN promoter; an immediate-early gene in combination with a baculovirus delayed-early gene promoter region selected from the group consisting of a 39K and a HindIII fragment containing a delayed-early gene; or a baculovirus late gene promoter. The immediate-early or delayed-early promoters can be enhanced with transcriptional enhancer elements.

Particularly suitable for use herein is the strong polyhedrin promoter of the baculovirus, which directs a high level of expression of a DNA insert, as described in Friesen et al. (1986) “The Regulation of Baculovirus Gene Expression” in: THE MOLECULAR BIOLOGY OF BACULOVIRUSES (W. Doerfler, ed.); EP 127,839 and EP 155,476; and the promoter from the gene encoding the plO protein, as described in Vlak et al., J. Gen. Virol. (1988) 69:765-776.

The plasmid for use herein usually also contains the polyhedrin polyadenylation signal, as described in Miller et al., Ann. Rev. Microbiol. (1988) 42:177 and a pr caryotic ampicillin-resistance (amp) gene and an origin of replication for selection and propagation in E. coli. DNA encoding suitable signal sequences can also be included and is generally derived from genes for secreted insect or baculovirus proteins, such as the baculovirus polyhedrin gene, as described in Carbonell et al., Gene (1988) 73:409, as well as mammalian signal sequences such as those derived from genes encoding human α-interferon as described in Maeda et al., Nature (1985) 315:592-594; human gastrin-releasing peptide, as described in Lebacq-Verheyden et al., Mol. Cell. Biol. (1988) 8: 3129; human IL-2, as described in Smith et al., Proc. Natl. Acad. Sci. USA (1985) 82:8404; mouse IL-3, as described in Miyajima et al., Gene (1987) 58:273; and human glucocerebrosidase, as described in Martin et al., DNA (1988) 7:99.

Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori host cells have been identified and can be used herein. See, for example, the description in Luckow et al., Bio/Technology (1988) 6: 47-55, Miller et al., in GENETIC ENGINEERING (Setlow, J. K. et al. eds.), Vol. 8 (Plenum Publishing, 1986), pp. 277-279, and Maeda et al., Nature, (1985) 315: 592-594. A variety of such viral strains are publicly available, e.g., the L-1 variant of Autographa californica NPV and the Bm-5 strain of Bombyx mori NPV. Such viruses may be used as the virus for transfection of host cells such as Spodoptera frugiperda cells.

Other baculovirus genes in addition to the polyhedrin promoter may be employed to advantage in a baculovirus expression system. These include immediate-early (alpha), delayed-early (beta), late (gamma), or very late (delta), according to the phase of the viral infection during which they are expressed. The expression of these genes occurs sequentially, probably as the result of a “cascade” mechanism of transcriptional regulation. Thus, the immediate-early genes are expressed immediately after infection, in the absence of other viral functions, and one or more of the resulting gene products induces transcription of the delayed-early genes. Some delayed-early gene products, in turn, induce transcription of late genes, and finally, the very late genes are expressed under the control of previously expressed gene products from one or more of the earlier classes. One relatively well defined component of this regulatory cascade is IEI, a preferred immediate-early gene of Autographo californica nuclear polyhedrosis virus (AcMNPV). IEI is pressed in the absence of other viral functions and encodes a product that stimulates the transcription of several genes of the delayed-early class, including the preferred 39K gene, as described in Guarino and Summers, J. Virol. (1986) 57:563-571 and J. Virol. (1987) 61:2091-2099 as well as late genes, as described in Guanno and Summers, Virol. (1988) 162:444451.

Immediate-early genes as described above can be used in combination with a baculovirus gene promoter region of the delayed-early category. Unlike the immediate-early genes, such delayed-early genes require the presence of other viral genes or gene products such as those of the immediate-early genes. The combination of immediate-early genes can be made with any of several delayed-early gene promoter regions such as 39K or one of the delayed-early gene promoters found on the HindIII fragment of the baculovirus genome. In the present instance, the 39 K promoter region can be linked to the foreign gene to be expressed such that expression can be further controlled by the presence of IEI, as described in L. A. Guarino and Summers (1986a), cited above; Guarino & Summers (1986b) J. Virol., (1986) 60:215-223, and Guarino et al. (1986c), J. Virol. (1986) 60:224-229.

Additionally, when a combination of immediate-early genes with a delayed-early gene promoter region is used, enhancement of the expression of heterologous genes can be realized by the presence of an enhancer sequence in direct cis linkage with the delayed-early gene promoter region. Such enhancer sequences are characterized by their enhancement of delayed-early gene expression in situations where the immediate-early gene or its product is limited. For example, the hr5 enhancer sequence can be linked directly, in cis, to the delayed-early gene promoter region, 39K, thereby enhancing the expression of the cloned heterologous DNA as described in Guarino and Summers (1986a), (1986b), and Guarino et al. (1986).

The polyhedrin gene is classified as a very late gene. Therefore, transcription from the polyhedrin promoter requires the previous expression of an unknown, but probably large number of other viral and cellular gene products. Because of this delayed expression of the polyhedrin promoter, state-of-the-art BEVs, such as the exemplary BEV system described by Smith and Summers in, for example, U.S. Pat. No., 4,745,051 will express foreign genes only as a result of gene expression from the rest of the viral genome, and only after the viral infection is well underway. This represents a limitation to the use of existing BEVs. The ability of the host cell to process newly synthesized proteins decreases as the baculovirus infection progresses. Thus, gene expression from the polyhedrin promoter occurs at a time when the host cell's ability to process newly synthesized proteins is potentially diminished for certain proteins such as human tissue plasminogen activator. As a consequence, the expression of secretory glycoproteins in BEV systems is complicated due to incomplete secretion of the cloned gene product, thereby trapping the cloned gene product within the cell in an incompletely processed form.

While it has been recognized that an insect signal sequence can be used to express a foreign protein that can be cleaved to produce a mature protein, the present invention is preferably practiced with a mammalian signal sequence appropriate for the gene expressed.

An exemplary insect signal sequence suitable herein is the sequence encoding for a Lepidopteran adipokinetic hormone (AKH) peptide. The AKH family consists of short blocked neuropeptides that regulate energy substrate mobilization and metabolism in insects. In a preferred embodiment, a DNA sequence coding for a Lepidopteran Manduca sexta AKH signal peptide can be used. Other insect AKH signal peptides, such as those from the Orthoptera Schistocerca gregaria locus can also be employed to advantage. Another exemplary insect signal sequence is the sequence coding for Drosophila cuticle proteins such as CP1, CP2, CP3 or CP4.

Currently, the most commonly used transfer vector that can be used herein for introducing foreign genes into AcNPV is pAc373. Many other vectors, known to those of skill in the art, can also be used herein. Materials and methods for baculovirus/insect cell expression systems are commercially available in a kit form from companies such as Invitrogen (San Diego Calif.) (“MaxBac” kit). The techniques utilized herein are generally known to those skilled in the art and are fully described in Summers and Smith, A MANUAL OF METHODS FOR BACULOVIRUS VECTORS AND INSECT CELL CULTURE PROCEDURES, Texas Agricultural Experiment Station Bulletin No. 1555, Texas A&M University (1987); Smith et al., Mol. Cell. Biol. (1983) 3: 2156, and Luckow and Summers (1989). These include, for example, the use of pVL985 which alters the polyhedrin start codon from ATG to ATT, and which introduces a BamHI cloning site 32 basepairs downstream from the ATT, as described in Luckow and Summers, Virology (1989) 17:31.

Thus, for example, for insect cell expression of the present polypeptides, the desired DNA sequence can be inserted into the transfer vector, using known techniques. An insect cell host can be cotransformed with the transfer vector containing the inserted desired DNA together with the genomic DNA of wild type baculovirus, usually by cotransfection. The vector and viral genome are allowed to recombine resulting in a recombinant virus that can be easily identified and purified. The packaged recombinant virus can be used to infect insect host cells to express a desired polypeptide.

Other methods that are applicable herein are the standard methods of insect cell culture, cotransfection and preparation of plasmids are set forth in Summers and Smith (1987), cited above. This reference also pertains to the standard methods of cloning genes into AcMNPV transfer vectors, plasmid DNA isolation, transferring genes into the AcmMNPV genome, viral DNA purification, radiolabeling recombinant proteins and preparation of insect cell culture media. The procedure for the cultivation of viruses and cells are described in Volkman and Summers, J. Virol. (1975) 19:820-832 and Volkman, al., J. Virol. (1976) 19:820-832.

Expression in Amphibian Cells

Expression of libraries of candidates for the practice of the invention can be conducted in the oocytes of amphibians. One amphibian particularly useful for this purpose is Xenopus laevis because of the capacity of the oocytes of this animal to express large libraries. Expression systems for X. laevis and other amphibians is established and expression conducted as described in Lustig and Kirschner, PNAS (1995) 92: 6234-38, Krieg and Melton (1987) Meth Enzymol 155:397-415 and Richardson et al. (1988) Bio/Technology 6:565-570.

For construction of libraries using Xenopus laevis oocytes, Xenopus oocytes are injected with cRNA libraries of candidate factors. The cRNA libraries are from plasmid DNAs from small cDNA library pools from a source such as a cell line or an animal organ. The plasmid DNAs are in vitro transcribed to cRNA and then injected into the oocyte, as described in Lustig and Kirschner, Krieg and Melton and Richardson et al, cited previously. The oocyte is incubated overnight at 18° C. The next day the oocyte is placed in microwells in contact with responsive cells. The microwells are incubated at 37° C. for 30 minutes to 3 hours. Candidate stimulatory or inhibitory factors, ligands, antagonists, or transcription factors are then expressed and secreted by the oocytes.

Expression in Mammalian Cells

Typical promoters for mammalian cell expression of the polypeptides of the invention include the SV40 early promoter, the CMV promoter, the mouse mammary tumor virus LTR promoter, the adenovirus major late promoter (Ad MLP), and the herpes simplex virus promoter, among others. Other non-viral promoters, such as a promoter derived from the murine metallothionein gene, will also find use in mammalian constructs. Mammalian expression may be either constitutive or regulated (inducible), depending on the promoter. Typically, transcription termination and polyadenylation sequences will also be present, located 3′ to the translation stop codon. Preferably, a sequence for optimization of initiation of translation, located 5′ to the polypeptide coding sequence, is also present. Examples of transcription terminator/polyadenylation signals include those derived from SV40, as described in Sambrook et al. (1989), cited previously. Introns, containing splice donor and acceptor sites, may also be designed into the constructs of the present invention.

Enhancer elements can also be used herein to increase expression levels of the mammalian constructs. Examples include the SV40 early gene enhancer, as described in Dijkema et al., EMBO J. (1985) 4:761 and the enhancer/promoter derived from the long terminal repeat (LTR) of the Rous Sarcoma Virus, as described in Gorman et al., Proc. Natl. Acad. Sci. USA (1982b) 79:6777 and human cytomegalovirus, as described in Boshart et al., Cell (1985) 41:521. A leader sequence can also be present which includes a sequence encoding a signal peptide, to provide for the secretion of the foreign protein in mammalian cells. Preferably, there are processing sites encoded between the leader fragment and the gene of interest such that the leader sequence can be cleaved either in vivo or in vitro. The adenovirus tripartite leader is an example of a leader sequence that provides for secretion of a foreign protein in mammalian cells.

Once complete, the mammalian expression vectors can be used to transform any of several mammalian cells. Methods for introduction of heterologous polynucleotides into mammalian cells are known in the art and include dextran-mediated transfection, calcium phosphate precipitation, polybrene mediated transfection, protoplast fusion, electroporation, encapsulation of the polynucleotide(s) in liposomes, and direct microinjection of the DNA into nuclei. General aspects of mammalian cell host system transformations have been described by Axel in U.S. Pat. No. 4,399,216.

The mammalian host cells used as responsive cells or producing cells in the invention may be cultured in a variety of media. Commercially available media such as Ham's F10 (Sigma), Minimal Essential Medium ([MEM], Sigma), RPMI-1640 (Sigma), and Dulbecco's Modified Eagle's Medium ([DMEM], Sigma) are suitable for culturing the host cells. In addition, any of the media described in Ham and Wallace, Meth. Enz. (1979) 58:44, Barnes and Sato, Anal. Biochem. (1980) 102:255, U.S. Pat. Nos. 4,767,704, 4,657,866, 4,927,762, or 4,560,655, WO 90/103430, WO 87/00195, and U.S. RE No. 30,985, may be used as culture media for the host cells. Any of these media may be supplemented as necessary to create optimal conditions for the function of the cells according to the method of the invention, including supplementation as necessary with hormones and/or other growth factors such as insulin, transferrin, or epidermal growth factor, salts (such as sodium chloride, calcium, magnesium, and phosphate), buffers (such as HEPES), nucleosides (such as adenosine and thymidine), antibiotics (such as Gentamycin™ M drug), trace elements (defined as inorganic compounds usually present at final concentrations in the micromolar range), and glucose or an equivalent energy sourcerange). Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

Small molecule libraries are made as follows. A “library” of peptides may be synthesized and used following the methods disclosed in U.S. Pat. No. 5,010,175, (the '175 patent) and in PCT WO91/17823. In method of the '175 patent, a suitable peptide synthesis support, for example, a resin, is coupled to a mixture of appropriately protected, activated amino acids.

The method described in WO91/17823 is similar. However, instead of reacting the synthesis resin with a mixture of activated amino acids, the resin is divided into twenty equal portions, or into a number of portions corresponding to the number of different amino acids to be added in that step, and each amino acid is coupled individually to its portion of resin. The resin portions are then combined, mixed, and again divided into a number of equal portions for reaction with the second amino acid. Additionally, one may maintain separate “subpools” by treating portions in parallel, rather than combining all resins at each step. This simplifies the process of determining which peptides are responsible for any observed alteration of gene expression in a responsive cell.

The methods described in WO91/17823 and U.S. Pat. No. 5,194,392 enable the preparation of such pools and subpools by automated techniques in parallel, such that all synthesis and resynthesis may be performed in a matter of days.

A further alternative agents include small molecules, including peptide analogs and derivatives, that can act as stimulators or inhibitors of gene expression, or as ligands or antagonists. Some general means contemplated for the production of peptides, analogs or derivatives are outlined in CHEMISTRY AND BIOCHEMISTRY OF AMINO ACIDS, PEPTIDES, AND PROTEINS—A SURVEY OF RECENT DEVELOPMENTS, Weinstein, B. ed., Marcell Dekker, Inc., publ. New York (1983). Moreover, substitution of D-amino acids for the normal L-stereoisomer can be carried out to increase the half-life of the molecule.

Peptoids, polymers comprised of monomer units of at least some substituted amino acids, can act as small molecule stimulators or inhibitors herein and can be synthesized as described in PCT 91/19735.. Presently preferred amino acid substitutes are N-alkylated derivatives of glycine, which are easily synthesized and incorporated into polypeptide chains. However, any monomer units which allow for the sequence specific synthesis of pools of diverse molecules are appropriate for use in producing peptoid molecules. The benefits of these molecules for the purpose of the invention is that they occupy different conformational space than a peptide and as such are more resistant to the action of proteases.

Peptoids are easily synthesized by standard chemical methods. The preferred method of synthesis is the “submonomer” technique described by R. Zuckermann et al., J. Am. Chem. Soc. (1992) 114:10646-7. Synthesis by solid phase techniques of heterocyclic organic compounds in which N-substituted glycine monomer units forms a backbone is described in copending application entitled “Synthesis of N-Substituted Oligomers” filed on Jun. 7, 1995 and is herein incorporated by reference in full. Combinatorial libraries of mixtures of such heterocyclic organic compounds can then be assayed for the ability to alter gene expression.

Synthesis by solid phase of other heterocyclic organic compounds in combinatorial libraries is also described in copending application U.S. Ser. No. 08/485,006 entitled “Combinatorial Libraries of Substrate-Bound Cyclic Organic Compounds” filed on Jun. 7, 1995, herein incorporated by reference in full. Highly substituted cyclic structures can be synthesized on a solid support by combining the submonomer method with powerful solution phase chemistry. Cyclic compounds containing one, two, three or more fused rings are formed by the submonomer method by first synthesizing a linear backbone followed by subsequent intramolecular or intermolecular cyclization as described in the same application.

Ribozymes and Antisense

Where the candidate capable of modulateing gene expression is a ribozyme, the ribozyme can be chemically synthesized or prepared in a vector for a gene therapy protocol including preparation of DNA encoding the ribozyme sequence. A ribozyme is a polynucleotide that has the ability to catalyze the cleavage of a polynucleotide substrate. Candidate ribozymes can be prepared and used as described in Long et al., FASEB J. 7: 25 (1993) and Symons, Ann. Rev. Biochem. 61: 641 (1992), Perrotta et al., Biochem. 31: 16, 17 (1992); and U.S. Pat. No. 5,225,337, U.S. Pat. No. 5,168,053, U.S. Pat. No. 5,168,053 and U.S. Pat. No. 5,116,742, Ojwang et al., Proc. Natl. Acad. Sci. USA 89: 10802-10806 (1992), U.S. Pat. No. 5,254,678 and in U.S. Pat. No. 5,144,019, U.S. Pat. No. 5,225,337, U.S. Pat. No. 5,116,742, U.S. Pat. No. 5,168,053. Preparation and use of such ribozyme fragments in a hammerhead structure are described by Koizumi et al., Nucleic Acids Res. 17:7059-7071(1989). Preparation and use of ribozyme fragments in a hairpin structure are described by Chowrira and Burke, Nucleic Acids Res. 20:2835 (1992).

The hybridizing region of the ribozyme or of an antisense polynucleotide may be modified by linking the displacement arm in a linear arrangement, or alternatively, may be prepared as a branched structure as described in Horn and Urdea, Nucleic Acids Res. 17:6959-67 (1989). The basic structure of the ribozymes or antisense polynucleotides may also be chemically altered in ways quite familiar to those skilled in the art.

Chemically synthesized ribozymes and antisense molecules can be screened as synthetic oligonucleotide derivatives modified by monomeric units. Ribozymes and antisense molecules can also be placed in a vector and expressed intracellularly for a screening assay.

The invention can be practiced by first determining the source of candidate factors to be screened. If the candidate factors are from a small molecule library, the factors are pooled appropriately, for example for testing in a 96-micro well format. If the candidate factors are encoded in cDNA or cRNA, a host cell system is selected, and after preparation of the appropriate cDNA or cRNA pools, the cells are injected or transformed with the pools of nucleic acid sequences. Where the candidate factors are expected to be secreted, the “producing” cells are co-cultured with the “responding” cells. Where the candidate factors may not be secreted, the host cell is lysed and the cell lysate is added to a microwell containing responding cells.

Where transcriptional changes provide the marker for identifying a positive factor, an appropriate transcriptional target is selected for screening the candidate factors for ability to transcriptionally modulate that target. This target can be any marker appropriate for detecting a desired response. So that, for example, where the desired response is a reduction of inflammation, the marker can be NFKB elements, and the signal can be a reduction in the level of transcript of such elements. In addition, where the desired response is an anti-tumor effect, the marker can be an oncogene, and the signal can be a reduction in the level of transcript of such an oncogene.

One particular advantage of the invention is the ability to screen a responding cell population without having to transform the cells with a reporter gene construct for detecting a factor. Thus, a primary cell culture can be screened. So that, for example, where whether a particular factor will have positive effect on a patient having a tumor can be tested in advance of a full therapeutic administration by removing some of the patient's cells and administering an appropriate dose of the therapeutic factor to the primary cell culture, and measuring the transcriptional or translational effects that ensue as a result of the therapeutic agent administration. Additionally, diagnosis can be made with a primary cell culture, where an anticipated transcriptional or translational change is expected in a disease, as compared to the transcription or translation levels of a target in a normal patient, and the patient's cells can be tested for the disease state levels of the transcript or translation product. For example, bDNA can be used to screen a population of patient cells against a known target gene transcript, where expression of the transcript indicates a disease state in the patient.

The invention can be practiced with such transformed responding cells, however, and the reporter gene can be used as a bDNA or PCR target, or as a reporter system in itself. Alternatively, a cell known to express the transcript of the target gene can be used as a responding cell, and after contact with a potentially modulatory factor, the up or down regulation of the target transcript can be measured directly by nucleic acid hybridization detection means, for example bDNA detection means, reverse transcription-polymerase chain reaction (RT-PCR) detection means, or RNAse protection assay means.

Other direct detection means are also available that would use the binding interaction between the nucleic acid of the transcript, and either a DNA, RNA, chemical, synthetic combination molecule (of more than one moiety such as a nucleic acid, chemical or amino acid moiety), or protein molecule capable of binding to it and also capable of subsequent detection as a bound pair. The detection is also possible, along similar lines, for a translation product, where the binding interaction is a protein binding interaction with either a DNA, RNA, chemical, synthetic combination molecule (of more than one moiety such as a nucleic acid, chemical or amino acid moiety) or protein detection molecule or probe. Some exemplary detection molecules are described in pending U.S. patent application Ser. No. 08/478,085, U.S. Pat. Nos. 5,451,503, 5,545,730, 5,541,313, 5,437,977, 5,430,138, 5,430,136, 5,424,413, 5,367,512, 5,124,246, 5,082,935, and 5,079,151, all incorporated by reference in full. Also included for examplary purposes is the information regarding detection molecules included in WO 96/06104 and EP 544 212, also incorporated by reference in full.

In cloning secreted growth factors the method of the invention was practiced selecting factor producing cells of either Xenopus oocytes or Cos cells, and factor response cells as either NIH3T3, PC12, or Hela-NFκβluc cells. Small pools of human and mouse cDNA libraries were introduced into the factor producing cells and factor response cells were cultured with supernatants collected from the factor producing cells or the factor producing cells were cocultured with the response cells. After a short period of time, the level of the immediate early genes, including gene products or mRNA transcribed from the gene, was indicated by an increase in c-fos transcription. Detection of the c-fos transcripts was accomplished by bDNA assay or luciferase reporter assay. A significant induction of a targeted immediate early gene, such as c-fos indicated that a particular subpool of cDNA corresponding to a particular sample was positive. The nature of the positive single clone was ulimately determined by sequencing. In using bDNA to detect the transcriptional event, bDNA was directed to c-fos transcript. When using a luciferase reporter construct, bDNA could also be used against the luciferase transcript, or the luciferase reporter activity could be detected.

Usually deconvolution was performed through two rounds. The first round of deconvolution was performed by first picking 1× (1×96) to 3× (3×96) clones of the average diversity (if 96) of the positive pool, and these clones were grown in the wells of 96-well plates. The cultures of each row of 96-well plates were pooled and their mini-pre DNAs or cRNAs were prepared and introduced into the factor producing cells. Single clones from the positive pool identified during the first round deconvolution were used for the second round deconvolution to identify a final positive single clone.

Manual defolliculated Xenopus oocytes are excellent at expressing in vitro transcribed cRNA and its background c-fos induction activity is almost none. Taking advantage of the size of Xenopus oocytes, a coculture system using Xenopus oocytes and NIH3T3 cells was developed in microplates which allowed the detection of both diffusible and membrane-attached nondiffusible growth factors. However, it was shown that damaged oocytes produce significant c-fos induction in NIH3T3 cells. A repeatable condition was established, however, which was sensitive enough to detect PDGF c-sis activity secreted from an oocyte injected with 0.08 ng of PDGF c-sis cRNA. This sensitivity was shown allowing for detection of PDGF c-sis activity in a pool of 600 clones. Screening was then conducted of 350 pools of a mouse brain cDNA library with a diversity of an average of 150 clones per pool, and 100 pools of a Xenopus embryo library with a diversity of 80 clones per pool. Considering the biological variation between oocytes, each pool of cRNA was injected into three oocytes and subsequently yielded triplicate results.

Among these 450 pools, 61 pools were initially found positive. After multiple repeated experiments, 14 remained moderately positive. Deconvolution of 3 pools, and partial deconvolution of 11 pools, yielded successful deconvolution of pool 16.1 to a single molecule, while deconvolution of the others (either completely or partially) resulted in loss of activity. No step-wise gain of c-fos inducibility was shown for pool 16.1, but the kinetics of 16.1 were similar to those of insulin-like growth factor (IGF). The nucleotide and translated protein sequence for clone 16.1 was determined, and is represented in SEQ ID NO. 1 (nucleotide) and SEQ ID NO. 2 (amino acid), indicating a 61 amino acid polypeptide of the sequence. The peptide 16.1 showed a 5 fold induction of c-fos transcription, as compared to a 30 fold induction seen with a FGF, demonstrating the feasibility of indentifying growth factors of both mild and pronounced transcriptional effects by the method of the invention.

A Cos cell/NIH3T3 system was developed for cloning diffusible growth factors, using a PDGF c-sis construct. The supernatants of the transfected Cos cells were assayed on NIH3T3 cells for c-fos inducibility by bDNA assay. The Cos cell system proved very efficient at expressing PDGF c-sis, and further the Cos cell supernatant did not carry a high background of c-fos induction. Activity was detectable at a diversity of a pool of 300 clones. A hundred pools (150 clones per pool) of a mouse brain cDNA library were screened, and 100 pools (100 clones per pool) of a mouse embryo library, and 400 pools (35-50 clones per pool) of a size-fractionated mouse embryo library were screened. Six positive pools from the mouse brain library and 1 positive pool from the mouse embryo library resulted.

Deconvolution of the first positive pool from the mouse brain library showed step-wise increase of c-fos inducibility. The sequence of the clone encoded a full-length bFGF cDNA, proving the principle of this work. The remaining 5 positive pools also indicated FGF as the growth factor involved.

There was an indication that one of the clones that had an FGF sequence, also had a non-FGF c-fos inducible clone, but the sequence indicated that no insert was present. Due to the deletion of the polyadenylation site, the clone potentially encodes an artificial peptide sequence capable of mimicing the function of a biological ligand. Among the 700 base pairs sequence after the RNA initiation site, two potential peptides (of 13 amino acids, and 22 amino acids) were found. Thus, the invention can be adapted to screen for artificial peptide sequences having biological activity.

By the method of the invention, Cos cells can be used as producing cells, and Cos cell supernatants can be tested for c-fos inducibility in PCl2 cells. Additionally, as part of a strategy to clone tumor necrosis factor (TNF)-like factors, a stable Hela cell line expressing luciferase gene under NFκβ element regulatory control, and thus responsive to TNF induction was designed. The luciferase assay and bDNA assay for luciferase mRNA both peaked at 4 hours after induction and yielded 20 fold induction of luciferase mRNA and protein over background. The luciferase assay was selected as the method of detection because of its convenience. Three hundred pools (35-50 clones per pool) of the size-fractionated mouse embryo library was screened for positives.

The inventors have confirmed the advantages of using a bDNA system for identification of the factors that modulate gene expression, over the standard thymidine incorporation assay. Parallel experiments were performed comparing a c-fos bDNA assay for measuring transcription and a thymidine incorporation assay for measuring cell proliferation. Thymidine incorporation measures DNA synthesis in cells. As a measure of DNA synthesis, labeled thymidine can be detected in the cells about 12 to 14 hours after thymidine admininstration. However, Cos supernatants have factors that inhibit growth, and can interefer with such a growth-based assay. For the comparative experiement, Cos supernatants were collected from cells transfected with PDGF c-sis DNA. Because transcription is an event that occurs relatively soon after a modulatory factor has contacted a cell, detecting transcriptional changes is possible, even where DNA synthesis can be ultimately inhibited by the supernatant. This is because detection of transcription can occur promptly after the modulatory effects have been exerted. In the comparative experiment, the Cos supernatants inhibited cell proliferation while retaining an ability for inducing c-fos mRNA in NIH3T3 cells. Thus, transcriptional detection, using, for example bDNA detection means, is superior to the thymidine incorporation assay for characterizing new growth factors.

Further objects, features, and advantages of the present invention will become apparent from the following detailed description. It should be understood, however, that the detailed description, while indicating preferred embodiments of the present invention, is given by way of illustration only, since various changes and modifications within the spirit and scope of the invention will become apparent to those skilled in the art from this detailed description. The invention is also not limited to any theories of action of the elements of the invention.

### EXAMPLE 1

### SRE-Luciferase Assay for SRE Inducible Transcription Factors

A construct comprising in operable linkage a luciferase reporter gene and the serum responsive element (SRE) was stably transfected into NIH3T3 derived FTL cells which were selected as the responsive cells. Plasmid DNAs from small library pools of 100 to 500 independent colonies per pool were constructed and in vitro transcribed into cRNAs. Each pool of cRNAs were injected into Xenopus laevis oocytes and cultured at 18° C. overnight. The next day, each oocyte was then cocultured with FTL cells transfected with the SRE-luciferase construct and incubated at 37° C. for three hours in a microwell. The oocytes were removed and induction of luciferase gene was assayed by luciferase assay, and also by bDNA assay against luciferase mRNA. The luciferase assay was sensitive to 4000 cells with a minimum concentration of 1 nM of PDGFBB (PDGF β chain dimer). The bDNA assay detected a lower concentration of library, with the ability to detect from 4000 cells less than 0.01 nM of PDGFBB.

### EXAMPLE 2

### Assay for A Factor that Acts at the CT Box Enhancer

An embodiment of the invention that is a variation on Example 1 above, can be conducted for screening for a stimulatory factor, including a polypeptide or a small molecule, that acts at the CT Box Enhancer taken from the insulin gene promoter by linking the CT box enhancer element from the insulin gene to the luciferase reporter gene. The rest of the experiment is conducted as described for example 1.

### EXAMPLE 3

### Assay for Growth Factor

To assay for a growth factor that stimulates the expression of the c-fos gene, a library is prepared from human tissue as described above for expression in Xenopus oocytes. A single oocyte transformed with a different library is placed in each microwell on a bed of PC12 cells, a progenitor cell line for nuerons. The system of the oocyte producing cells and the PC12 responsive cells is cultured at 37° C. for 30 minutes to 3 hours. The wells are assayed with bDNA probes to c-fos mRNA. The positive pools are divided into subpools and the assay conducted again until a single factor responsible for the upregulation of c-fos is identified.

### EXAMPLE 4

### Assay for a Differentiation Factor

PC12 cells are cultured with the supernatant of Xenopus oocytes injected with a cRNA library for 3 to 5 days. The supernatant is used during the incubation because an oocyte would not withstand the length of incubation time necessary for response to a differentiation factor. The PC12 cells are then observed under a microscope for neurite outgrowth phenotype.

### EXAMPLE 5

### Assay for an Inhibitor

Where an inhibitor from a small molecule library is sought, a small molecule peptoid library is prepared according to methods known in the art. Jurkat cells that express IL-2 are selected as the responsive cells. The Jurkat cells are incubated in the presence of pools of the peptoid library, and assayed by bDNA against the IL-2 transcript. Positives are identified by a down-regulation of transcription of IL-2 compared to Jurkat cells not exposed to the library. Peptoids that cause a down-regulation of IL-2 are further characterized for their potential immunosuppresant capability.

### EXAMPLE 6

### Assay for The Wnt Receptor from Wingless Drosophilia

Clone-8 drosophila imaginal disc cells are cultured with Wingless protein and assayed by differential mRNA and PCR to identify a gene transcriptionally responsive to Wingless. From the sequence of this gene, a bDNA probe is made. A cDNA library is made from clone-8 cells and a cRNA library copy injected into Xenopus laevis oocytes and cultured. bDNA probes are used to screen the library and the isolation of positive pools proceeds until a single clone is identified.

### EXAMPLE 7

### Assay for an Ob-Inducing Compound

Ob-inducing molecules can be screened from small molecule libraries, or from cell, tissue, or plant extracts. The responsive cells are adipocytes that express the ob gene. bDNA for the ob gene transcript is used to detect positives, and the assay procedes until a subpool contains a single factor responsible for the positive.

### EXAMPLE 8

### Cloning Growth Factors by Measuring Induction of Endogenous c-fos Messengers

To assay for a growth factor that stimulates the expression of the c-fos gene, NIH3T3 derived FTL cells were selected as the responsive cells. A library for screening is prepared from human tissue as described above for expression in Xenopus oocytes. Plasmid DNAs from small library pools of 100 to 500 independent colonies per pool were constructed and in vitro transcribed into cRNAs. Each pool of cRNAs were injected into Xenopus laevis oocytes and cultured at 18° C. overnight. The next day, each oocyte was then cocultured on a bed of NIH3T3 derived FTL cells in a microwell and incubated at 37° C. for three hours.

The oocytes were removed and induction of c-fos messenger was measured by bDNA assay against c-fos mRNA. In a previous study (described in example 1) an SRE-luciferase reporter gene construct transfected into FTL cells was shown to be sensitive to 4000 cells with a minimum concentration of 1 nM of PDGFBB (PDGF β chain dimer). In the present experiment, a bDNA assay detected a lower concentration of library, with the ability to detect from 4000 FTL cells less than 0.01 nM of PDGFBB (PDGF β chain dimer). As previously described, administration of PDGFBB to cells, including FTL cells, induces the transcription of the c-fos gene. Thus, the sensitivity of the bDNA assay indicates that even with a very low concentration of growth factor, growth factor induction of c-fos can be detected, and the growth factor then identified and cloned by the method of the invention.

### EXAMPLE 9

### Screen of Small Molecule Libraries for Antagonists and Agonists of c-fos Induction

Small molecule library pools are prepared as described above, and 48 such pools were marked and divided for screening. An aliquot of each pool (2 μM final concentration) was placed in a micro-well on a bed of NIH3T3 cells in a microwell and incubated at 37° C. for 45 minutes with 1 ηM PDGFBB.

Each well was assayed by induction of c-fos messenger by bDNA assay against c-fos mRNA. In a previous study (described in example 8), a bDNA assay detected induction of c-fos mRNA by less than 0.01 ηM of PDGFBB (PDGF β chain dimer). 1 ηM of PDGFBB gives 50% of maximal c-fos induction. As previously described, administration of PDGFBB to cells, including FTL cells, induces the transcription of the c-fos gene.

In this study, induction or reduction of the c-fos gene was measured by bDNA. Where an agonist was present in the small molecule library pool, c-fos induction was expected to occur at about the same level or greater as induction by administration of PDGFBB. Where an antagonist was present in the small molecule library pool, c-fos transcription was expected to be reduced perhaps to non-detectable levels. With the confidence gathered from the demonstration in example 8 of the sensitivity of the bDNA assay, only a very low amount of growth factor receptor agonist or antagonist small molecule was needed for this type of screening.

Small molecule pools were then screened using the standard for antagonist pools as more than 50% reduction of transcription levels compared to the c-fos level induced by 1 ηM PDGFBB, and for agonist pools as more than 50% increase of transcription levels of c-fos levels compared with that induced by 1ηM PDGFBB.

In the present study, of 48 pools of small molecules, 2 potential agonists and 5 potential antagonists have been identified, and are being isolated by successive screenings to determine the specific small molecule responsible for the control of c-fos transcription.

The 48 small molecule pools are heterocyclic mixture pools. The standard used for this experiment for antagonist pools is more than 50% reduction compared to c-fos level induced by 1 ηM PDGFBB, and for agonist pools is more than 50% increase of c-fos level compared with that induced by 1 ηM PDGFBB.

### EXAMPLE 10

### Identification of a 61 Amino Acid Polypeptide Capable of Inducing c-fos Transcription in NIH3T3 Cells

Xenopus oocytes were injected with cRNA, in triplicate (the same pools were injected 3 times into 3 different oocytes) for expression of candidate growth factors, using the methods described in Example 8. The sensitivity of the responsiveness of the responding cells, NIH3T3 cells was tested and designed to detect PDGF c-sis activity secreted from an oocytes injected with 0.08 ng of PDGF c-sis cRNA, in a pool of 600 clones. A mouse brain library was divided into 350 pools, with a diversity average of 150 clones per pool, as well as 100 pools of a Xenopus embryo library with a diversity of 80 clones per pool. Among the 450 pools, 61 pools were initially found positive, and one was deconvoluted to a single molecule, capable of c-fos induction of about 5-fold as compared to non-stimulated NIH3T3 cells. The nucleotide and amino acid sequences of this clone are embodied in SEQ ID NO. 1, and 2 respectively.

### EXAMPLE 1

### Cos/NIH3T3 System Identifying Known Diffusible Growth Factor FGF

Using a PDGF c-sis construct, a Cos cell system for efficiently expression PDGF c-sis was developed. The supernatants of the transfected Cos cells were assayed on NIH3T3 cells for the ability of the supernatant to induce c-fos as detected by bDNA assay against c-fos transcript, as described earlier. The Cos cells were shown to be very efficient at expressing PDGF c-sis. Further, the Cos cell system allowed detection of c-fos activity at a diversity of a pool of 300 clones. One hundred pools (of 150 clones per pool) of a mouse brain cDNA library, 100 pools (of 100 clones per pool) of a mouse embryo library, and 400 pools (of 35-50 clones per pool) of a size-fractionated mouse embryo library were screened. Six positive pools were identified from the mouse brain library were identified.

The deconvolution of the first positive pool (100.3) from the mouse brain library showed step-wise increase of c-fos inducibility. The sequence of the deconvoluted final clone of 100.3 showed that it encoded a full-length bFGF (fibroblast growth factor) cDNA. The cloning of FGF through Cos/NIH3T3 system demonstrates the potential of the system. The remaining 5 positive pools of the mouse embryo library were also deconvoluted to bFGF cDNA. FGF is a growth factor known to induce c-fos transcription.

### EXAMPLE 12

### Testing B-cells Responsiveness to Candidate Therapeutic Agents For Treating B-cell Lymphoma

Ten ml of heparinized peripheral blood is obtained from a patient having B-cell lymphoma. The blood is layered onto 10 ml of Ficoll-Hypaque at a density of 1.077 g/ml and spun at 1900× g for 15 minutes at 20° C. The white cells are removed at the interface, and washed 2× in sterile phosphate buffered saline. The cells are resuspended and plated for 60 minutes at 37° C. to allow the monocytes to adhere. The non-adherent cells are removed and mixed with an equal volume of sheep erythrocytes that have been treated with 2-aminoethylisothioruonium bromide hydrobromide in a flat-bottomed steril glass bottle. The cells are spun at 300× g for 10 minutes at 20° C. and let stand for 2 hours at room temperature. The resuspended cells are gently suspended and the rosetted T-cells removed by layering onto Ficoll-Hypaque (10 ml). The white cells are removed from the interface, which are primarily B-cells, and resuspended and washed in 2× in phosphate buffered saline. Ten ml of blood should give about 106 cells from healthy donors, and variable amounts from patients with B-cell lymphoma. The cells are then plated in microwell plates.

Candidate therapeutics for the B-cells are tested in the wells, and screened for reduction in IL-2 expression upon administration of the therapeutics. bDNA specific for IL-2 transcript is used to detect reduction in IL-2 transcription.

What is claimed:

1. A method of high throughput screening for a factor that modulates gene expression comprising the steps:
(a) providing a small amount of a candidate factor,
(b) providing a small amount of responding cells,
(c) contacting a responding cell with a candidate factor, wherein the responding cell is capable of responding to a factor that modulates gene expression by exhibiting an early intracellular event; and
(d) detecting directly the early intracellular event.

2. The method of claim 1, wherein step (d) comprises a detection facilitated by hybridization of a polynucleotide sequence to a target polynucleotide.

3. The method of claim 2, wherein the target polynucleotide is an mRNA transcript.

4. The method of claim 2, further comprising that the polynucleotide sequence that hybridizes to a target polynucleotide is selected from the group consisting of synthetic DNA, synthetic RNA, reverse transcription primers, polymerase chain reaction primers, and an RNAse protection assay probe.

5. The method of claim 4, wherein the synthetic DNA comprises branched DNA.

6. The method of claim 1, wherein step (a) comprises a candidate factor selected from the group consisting of a polypeptide factor, small molecule factor, and a polynucleotide factor.

7. The method of claim 6, wherein the polypeptide factor is encoded by a cDNA or a cRNA molecule.

8. The method of claim 7, wherein the cDNA or cRNA molecule is expressed in a producing cell.

9. The method of claim 6, wherein the small molecule factor comprises a small molecule selected from the group consisting of a small organic molecule, a peptide molecule, and a peptoid molecule.

10. The method of claim 6, wherein the polynucleotide factor comprises one selected from the group consisting of a ribozyme and an antisense oligonucleotide.

11. The method of claim 1, wherein step (c) comprises co-culturing a producing cell and a responding cell.

12. The method of claim 11, wherein the producing cell is transformed with a cDNA or a cRNA encoding a candidate polypeptide factor.

13. The method of claim 11, wherein the producing cell is a Xenopus oocyte.

14. The method of claim 11, wherein the responding cell is a mammalian cell.

15. The method of claim 1, wherein the candidate factor that modulates gene expression comprises a factor selected from the group consisting of a stimulatory factor, and an inhibitory factor.

16. The method of claim 15, wherein the stimulatory factor comprises a factor selected from the group consisting of a growth factor, a transcription factor, a differentiation factor, a developmental regulator, an apoptotic factor, an immunomodulatory factor, and an oncogenic factor.

17. The method of claim 2, wherein the target polynucleotide comprises a transcript of a gene encoding an inducible protein.

18. The method of claim 2, wherein the target polynucleotide comprises a transcript of a gene encoding a protein selected from the group consisting of a cytokine, a hematopoetic factor, a neuronal differentiation factor, a growth factor, a protein hormone, a repressor protein, and a tissue marker.

19. The method of claim 17, wherein the target polypeptide comprises a transcript of a gene encoding a protein selected from the group consisting of a DNA-binding protein, a disease marker, a growth marker, a differentiation marker, an apoptotic marker, a metastatic marker, a marker associated with a later onset of a disease, and an oncogene.

20. The method of claim 1, wherein step (a) comprises that the candidate factor is selected from the group consisting of a natural product of a producing cell, a product of expression by a producing cell that is transformed with a polynucleotide sequence encoding the candidate factor, and a small molecule derived from a small molecule library.

21. The method of claim 1 wherein step (b) comprises responding cells that are natural cells.

22. The method of claim 1, wherein step (b) comprises responding cells that are transformed with a reporter gene construct.

23. The method of claim 8, wherein the candidate factor is expressed on the surface of the producing cell.

24. The method of claim 8, wherein the candidate factor is secreted by the producing cell.

25. The method of claim 1, wherein the responding cell comprises a prokaryotic cell or an eukaryotic cell.

26. The method of claim 25, wherein the responding cell comprises a eukaryotic cell selected from the group consisting of a mammalian cell, a fungal cell, an insect cell, an avian cell, a worm cell, a fish cell, a crustacean cell, a reptilian cell, an amphibian cell, and a plant cell.

27. The method of claim 8, wherein the producing cell comprises a prokaryotic cell or an eukaryotic cell.

28. The method of claim 27, wherein the producing cell comprises a eukaryotic cell selected from the group consisting of a mammalian cell, a fungal cell, an insect cell, an avian cell, a worm cell, a fish cell, a crustacean cell, a reptilian cell, an amphibian cell, and a plant cell.

29. The method of claim 1, wherein the early intracellular event of step (d) comprises an increase or decrease in transcription.

30. The method of claim 15, wherein the inhibitory factor decreases a target polynucleotide amount, wherein the target polynucleotide comprises a transcript of a gene encoding a protein selected from the group consisting of a cytokine, a hematopoetic factor, a neuronal differentiation factor, a growth factor, a protein hormone, a repressor protein, and a tissue marker.

31. The method of claim 30, wherein the target polypeptide comprises a transcript of a gene encoding a protein selected from the group consisting of a DNA-binding protein, a disease marker, a growth marker, a differentiation marker, an apoptotic marker, a metastatic marker, a marker associated with a later onset of a disease, and an oncogene.

32. The method of claim 1 for use in high throughput screening for a receptor that binds a known ligand comprising the modification of steps (a) through (b) of:
(a) providing a small amount of a candidate receptor,
(b) providing a responding cell transformed with a polynucleotide sequence encoding a known ligand,
(c) contacting a responding cell that comprises a candidate receptor with a ligand to allow formation of a receptor/ligand specific binding pair that triggers a detectable early intracellular event in the responding cell,
(d) detecting the early intracellular event.

33. The method of claim 32, wherein the ligand comprises a molecule selected from the group consisting of a natural product of a producing cell, a product of expression by a producing cell that is transformed with a polynucleotide sequence encoding the ligand, and a small molecule.

34. The method of claim 32, wherein the responding cell is transformed with a polynucleotide encoding the candidate receptor.

35. The method of claim 34, wherein the polynucleotide comprises one selected from the group consisting of a cDNA molecule, a cRNA molecule, and a genomic DNA molecule.

36. The method of claim 34, wherein the polynucleotide is derived from a cDNA library of a life form selected from the group consisting of a mammal, a fungus, an insect, a worm, a bird, a fish, a crustacean, a bacterium, a reptile, an amphibian, and a plant.

37. The method of claim 36, wherein the mammal is a human.

38. The method of claim 1, wherein step (d) comprises that the early intracellular event is detected by one selected from the group consisting of a bDNA assay, a RNase protection assay, and RT-PCR.

39. The method of claim 32, wherein the intracellular event is detected by one selected from the group consisting of a bDNA assay, a RNase protection assay, and RT-PCR.

40. The method of claim 32, wherein the known ligand comprises a ligand selected from the group consisting of Noggin, Wnt, and Notch.

41. The method of claim 1 for use in high throughput screening for an unknown ligand that binds a known receptor comprising the modification of steps (a) through (b) of:
(a) providing a small amount of a candidate ligand,
(b) providing a responding cell transformed with a polynucleotide sequence encoding a known receptor,
(c) contacting a responding cell that expresses a known receptor with a candidate ligand to allow formation of a receptor/ligand specific binding pair that triggers a detectable early intracellular event in the responding cell,
(d) detecting the early intracellular event.

42. The method of claim 41, wherein the ligand comprises a molecule selected from the group consisting of a natural product of a producing cell, a product of expression by a producing cell that is transformed with a polynucleotide sequence encoding the ligand, and a small molecule.

43. The method of claim 41, wherein the candidate ligand is an antagonist to a receptor.

44. The method of claim 41, wherein step (a) comprises transforming a producing cell with a polynucleotide encoding a candidate ligand.

45. The method of claim 44, wherein the polynucleotide comprises a molecule selected from the group consisting of a cDNA, a cRNA and a genomic DNA molecule.

46. The method of claim 45, wherein the cDNA or genomic DNA molecule is derived from a sequence selected from a library derived from the group consisting of a mammal, a fungus, an insect, a worm, a bird, a fish, a crustacean, a bacterium, a reptile, an amphibian, and a plant.

47. The method of claim 46, wherein the library is derived from a mammal.

48. The method of claim 47, wherein the mammal is a human.

49. The method of claim 41, wherein the intracellular event is detected by a assay capable of detecting changes in transcription of a gene.

50. The method of claim 49, wherein the assay comprises one selected from the group consisting of a bDNA assay, a RNase protection assay, and RT-PCR.

51. The method of claim 1 wherein the factor causes regulation of transcriptional activity of a gene.

52. The method of claim 51, wherein step (c) further comprises contacting a candidate factor to be tested for its ability to regulate transcriptional activity with a responding cell that comprises a regulatory sequence subject to transcriptional regulation, and further wherein step (d) further comprises detecting up-regulation or down-regulation activity of the regulatory sequence.

53. The method of claim 51, wherein the responding cell is capable of responding to a factor that modulates gene expression by exhibiting an early intracellular event.

54. The method of claim 51, wherein the responding cell is not transformed with a reporter gene sequence connected to a regulatory sequence subject to transcriptional regulation.

55. The method of claim 51, wherein the responding cell is transformed with a reporter gene sequence connected to a regulatory sequence subject to transcriptional regulation.

56. The method of claim 55, wherein the detection of up-regulation or down-regulation is performed by detection of reporter gene expression.

57. The method of claim 56, wherein the reporter gene comprises one selected from the group consisting of luciferase, secreted alkaline phosphatase, β-galactosidase, CAT, and GFP.

58. The method of claim 56, wherein the detection of reporter gene expression is accomplished by a nucleic acid hybridization assay.

59. The method of claim 57, wherein the reporter gene expression is detected by one selected from the group consisting of a bDNA assay, a RNase protection assay and RT-PCR.

60. The method of claim 52, wherein the regulatory sequence comprises one selected from the group consisting of a promoter, an enhancer and a repressor.

61. The method of claim 51, wherein the candidate factor comprises one selected from the group consisting of a natural product of a producing cell or virus, a product of expression by a producing cell or virus that is transformed with a polynucleotide sequence encoding the candidate factor, and a small molecule.

62. The method of claim 52, wherein the regulatory sequence comprises a regulatory sequence derived from a gene selected from the group consisting of a viral gene, a bacteriophage gene, a prokaryotic gene and an eukaryotic gene.

63. The method of claim 62, wherein the eukaryotic gene comprises one selected from the group consisting of cytokines, hematopoetic factors, neuronal differentiation factors, growth factors, differentiation factors, protein hormones, transcription factors, repressor proteins, DNA-binding proteins, tissue markers, cancer markers, disease markers, ob protein, A20 protein, ICAM, c-fos protein, and any inducible protein.

64. The method of claim 62, wherein the eukaryotic gene comprises one selected from the group consisting of a mammalian gene, a fungal gene, a worm gene, an insect gene, an avian gene, a fish gene, a crustacean gene, a reptilian gene, an amphibian gene, and a plant gene.

65. A growth factor discovered by the method of claim 1.

66. A differentiation factor discovered by the method of claim 1.

67. An inhibitory factor discovered by the method of claim 1.

68. A ligand discovered by the method of claim 1.

69. A receptor discovered by the method of claim 1.

70. A hormone discovered by the method of claim 1.

71. A cytokine discovered by the method of claim 1.

72. A transcription factor discovered by the method of claim 1.

73. An antagonist to a receptor discovered by the method of claim 1.

74. A polynucleotide comprising a nucleotide sequence encoding the growth factor of claim 65.

75. A polynucleotide comprising a nucleotide sequence encoding the differentiation factor of claim 66.

76. A polynucleotide comprising a nucleotide sequence encoding the inhibiting factor of claim 67.

77. A polynucleotide comprising a nucleotide sequence encoding the ligand of claim 68.

78. A polynucleotide comprising a nucleotide sequence encoding the receptor of claim 69.

79. A polynucleotide comprising a nucleotide sequence encoding the hormone of claim 70.

80. A polynucleotide comprising a nucleotide sequence encoding the cytokine of claim 71.

81. A polynucleotide comprising a nucleotide sequence encoding the transcription factor of claim 72.

82. A polynucleotide comprising a nucleotide sequence encoding the antagonist to a receptor of claim 73.

83. A polypeptide comprising a portion of the sequence of SEQ ID NO. 2, and exhibiting growth factor activity as demonstrated by induction of c-fos transcription.

84. The polypeptide of claim 83, wherein the induction of c-fos transcription is at least about 5-fold induction above normal levels.

85. A polypeptide having the sequence of SEQ ID No. 2.

86. A polypeptide comprising a molecule selected from the group consisting of an analog, a derivative, and a variant of the polypeptide of claim 85.

87. A polynucleotide sequence encoding the polypeptide of claim 86.

88. A polynucleotide sequence of SEQ ID No. 1 connected to a heterologous polynucleotide sequence.


SEQUENCE LISTING (2 sequences)

SEQ ID NO: 1 - DNA (cDNA), 448 bp; single-stranded, linear
acttgttctttttgcaggatcccatcgattcgaattcggcacgagggtaatgtatacgtaggcgatatatgtaagtgttctgataacatgactctaatggtgtttaagtgttctgataacctgcttttaatagttgtgtatgccaaagtctttcccagcacctcttgtattcattatcaaatatatacatgtaagtttgaagtattactgttttctcagcatgaattaaaaatattctgtaactcagccaggcaaggtagctcatgcctgtgatcccagcatttgggaaacagaggcaagaggattgctgcaagtttaaggccagcctgtctacatagggaggtccaggccagggatgttatatcaagatcttgtttcaaaaattatatatatatatatatatatatatatatatatatatatatatatatatatatatatatatata

SEQ ID NO: 2 - protein, 61 aa
MTLMVFKCSDNLLLIVVYAKVFPSTSCIHYQIYTCKFEVLLFSQHELKIFCNSARQGSSCL